(19) United States
(12) Patent Application Publication
(10) Pub. No.: US 2019/0088884 A1
(43) Pub. Date: Mar. 21, 2019

(54) COMPOUNDS FOR ORGANIC LIGHT EMITTING DIODE MATERIALS

(71) Applicant: President and Fellows of Harvard College, Cambridge, MA (US)

(72) Inventors: Alan Aspuru-Guzik, Cambridge, MA (US); Rafael Gomez-Bombarelli, Cambridge, MA (US); Timothy D. Hirzel, Quincy, MA (US); Jorge Aguilera-Iparraguirre, Roslindale, MA (US)

(21) Appl. No.: 16/091,844

(22) PCT Filed: Apr. 5, 2017
(86) PCT No.: PCT/US2017/026071

§ 371 Date: Oct. 5, 2018

Related U.S. Application Data
(60) Provisional application No. 62/318,531, filed on Apr. 5, 2016.

(51) Int. Cl. H01L 51/00; C07D 221/20; C07F 7/08; C09K 11/06

(52) U.S. Cl. CPC H01L 51/0072; C07D 221/20; H01L 51/5016; C09K 11/06; C09K 2211/1018; C07F 7/0816

(57) ABSTRACT

Described herein are molecules for use in organic light emitting diodes. Example compounds include molecules represented by structural formula (I). Values and example value of in the structural formula (I) are defined herein.

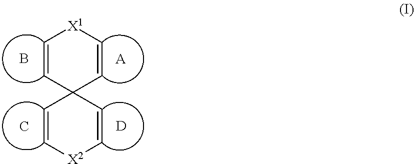

### RELATED APPLICATION

This application claims the benefit of U.S. Provisional Application No. 62/318,531, which was filed on Apr. 5, 2016. The entire teachings of this application are incorporated herein by reference.

### BACKGROUND OF THE INVENTION

An organic light emitting diode (OLED) is a light-emitting diode (LED) in which a film of organic compounds is placed between two conductors and emits light in response to excitation, such as an electric current. OLEDs are useful in displays such as television screen, computer monitors, mobile phones, and tablets. A problem inherent in OLED displays is the limited lifetime of the organic materials. OLEDs which emit blue light, in particular, degrade at a significantly increased rate as compared to green or red OLEDs.

OLED materials rely on the radiative decay of molecular excited states (excitons) generated by recombination of electrons and holes in a host transport material. The nature of excitation results in interactions between electrons and holes that split the excited states into bright singlets (with a total spin of 0) and dark triplets (with a total spin of 1). Since the recombination of electrons and holes affords a statistical mixture of four spin states (one singlet and three triplet sublevels), conventional OLEDs have a maximum theoretical efficiency of 25%.

To date, OLED material design has focused on harvesting the remaining energy from the normally dark triplets into an emissive state. Recent work to create efficient phosphors, which emit light from the normally dark triplet state, have resulted in green and red OLEDs. Other colors, such as blue, however, require higher energy excited states which enhance the degradation process of the OLED.

The fundamental limiting factor to the triplet-singlet transition rate is a value of the parameter |H_{fi}/Δ|², where H_{fi} is the coupling energy due to hyperfine or spin-orbit interactions, and Δ is the energetic splitting between singlet and triplet states. Traditional phosphorescent OLEDs rely on the mixing of singlet and triplet states due to spin-orbital (SO) interaction, increasing H_{fi} and affording a lowest emissive state shared between a heavy metal atom and an organic ligand. This results in energy harvesting from all higher singlet and triplet states, followed by phosphorescence (relatively short-lived emission from the excited triplet). The shortened triplet lifetime reduces triplet exciton annihilation by charges and other excitons. Recent work by others suggests that the limit to the performance of phosphorescent materials has been reached.

### SUMMARY OF THE INVENTION

Thus, a need exists for OLEDs which can reach higher excitation states without rapid degradation. It has now been discovered that thermally activated delayed fluorescence (TADF), which relies on minimization of Δ as opposed to maximization of H_{fi}, can transfer population between singlet levels and triplet sublevels in a relevant timescale, such as, for example, 110 μs. The compounds described herein are capable of fluorescing or phosphorescing at higher energy excitation states than compounds previously described.

Accordingly, in some embodiments, the present invention is a molecule represented by the following structural formula:

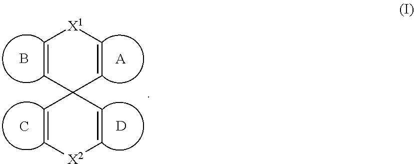

In structural formula (I) of the present invention:
X¹ and X² are independently selected from O, S, C(O), CR^{a}R^{b}, SiR^{a}R^{b}, NR^{c}, BR^{c}, or a bond. Rings A, B, C, and D are, each independently, optionally substituted aromatic or heteroaromatic rings. In some embodiments, at least one of rings A, B, C, and D contains at least one N.
Each instance of R^{a}, R^{b}, and R^{c} is independently selected from H, a C₁-C₆ alkyl, a C₃-C₁₈ cycloalkyl, a C₆-C₁₈ aryl, a 5-20 atom heteroaryl, halo, or —CN.

Accordingly, in some embodiments, the present invention is a molecule represented by the following structural formula:

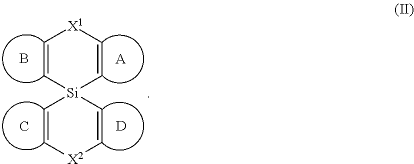

In structural formula (II) of the present invention:
X¹ and X² are independently selected from O, S, C(O), CR^{a}R^{b}, SiR^{a}R^{b}, NR^{c}, BR^{c}, or a bond. Rings A, B, C, and D are, each independently, optionally substituted aromatic or heteroaromatic rings. In some embodiments, at least one of rings A, B, C, and D contains at least one N.
Each instance of R^{a}, R^{b}, and R^{c} is independently selected from H, a C₁-C₆ alkyl, a C₃-C₁₈ cycloalkyl, a C₆-C₁₈ aryl, a 5-20 atom heteroaryl, halo, or —CN.

In some embodiments, the present invention is a molecule represented by one of the structural formulas in Table 1. In some embodiments, the present invention is represented by one of the structural formulas in Table 1, wherein any substitutable carbon is optionally substituted with R^{d}, and each R^{d} is independently selected from H, a C₁-C₆ alkyl, a C₁-C₁₈ cycloalkyl, a C₆-C₁₈ aryl, a 5-20 atom heteroaryl, halo, or —CN.

In some embodiments, the present invention is an organic light-emitting device comprising a first electrode, a second electrode, and an organic layer between the first electrode and the second electrode. The organic layer comprises at least one light-emitting molecule selected from structural formulas (I) or (II), or from the structural formulas in Table 1.

In a fourth embodiment, the present invention is a molecule represented by one of the following structural formulas:

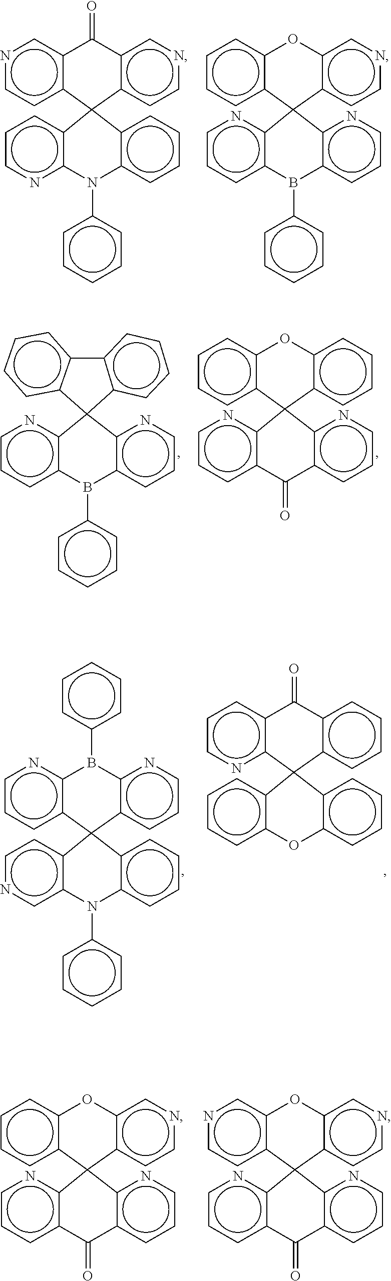

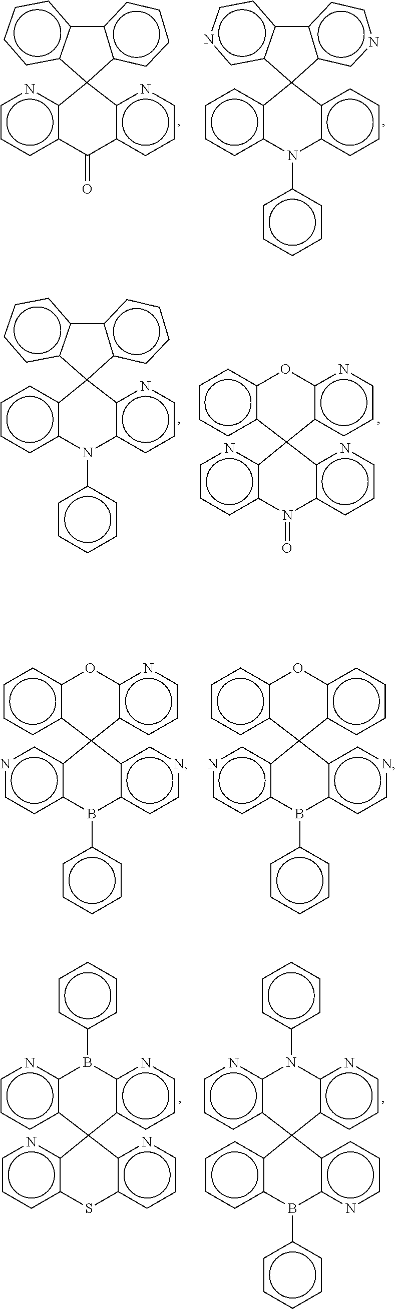

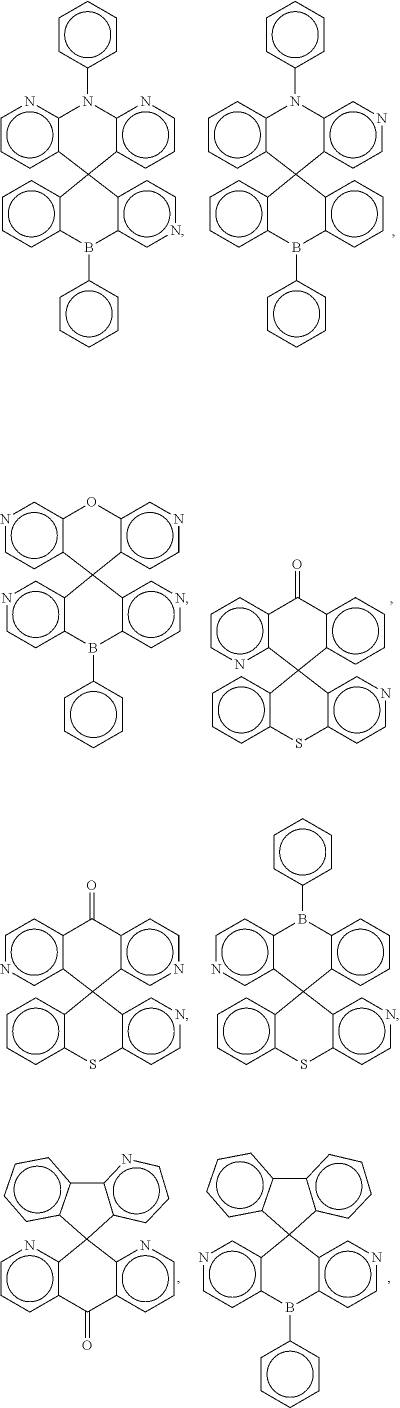

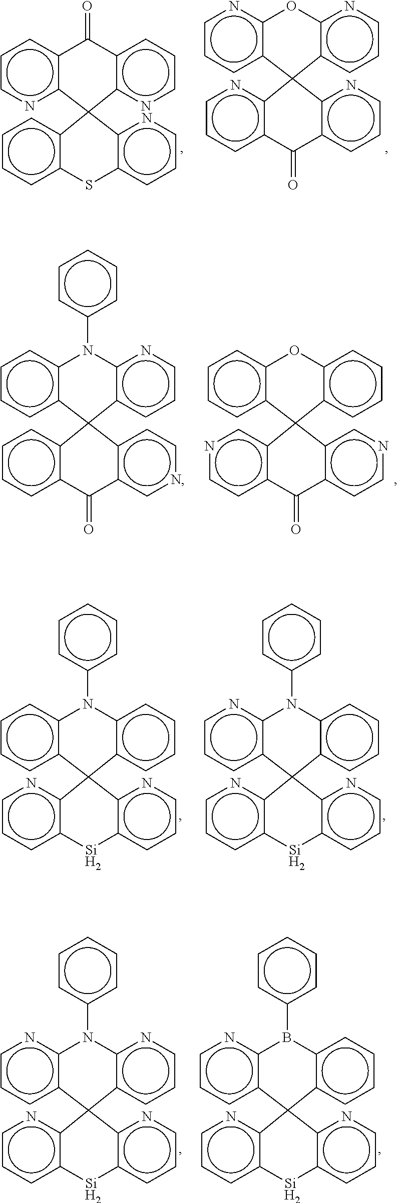

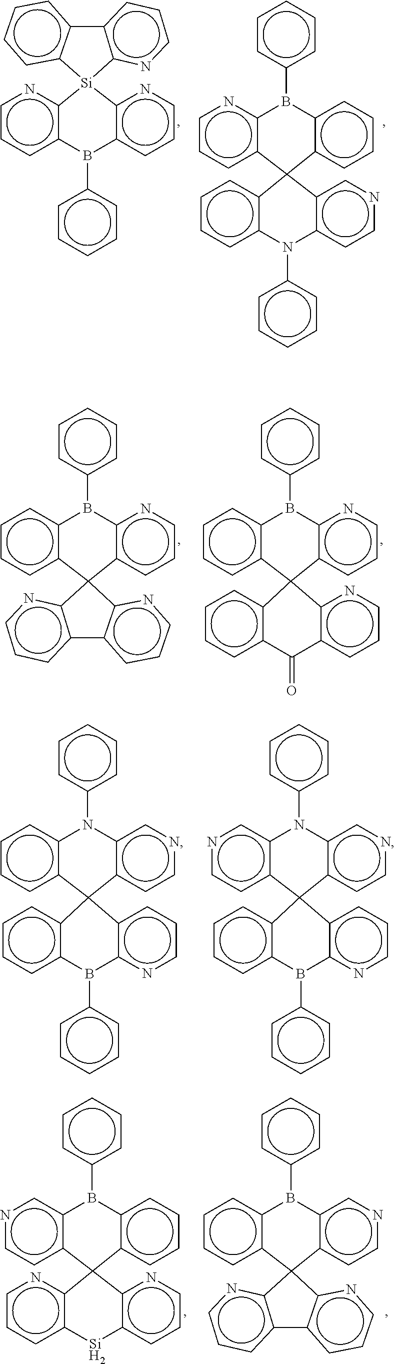

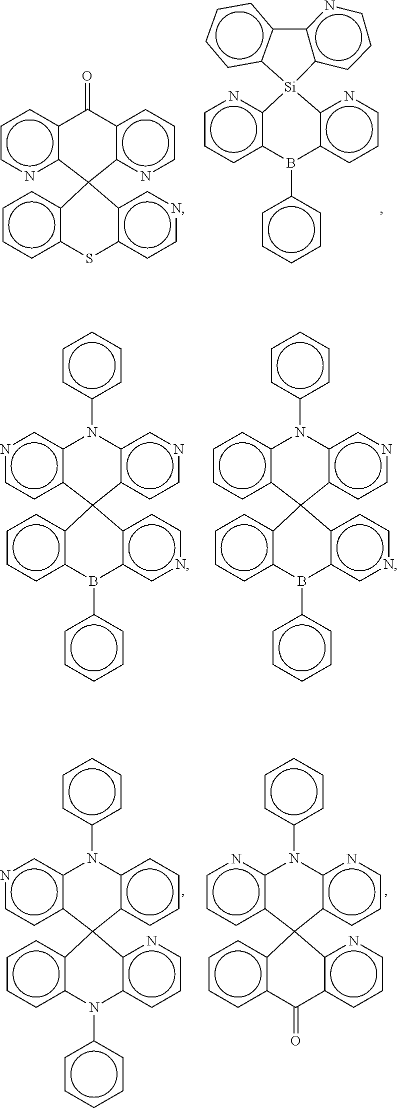

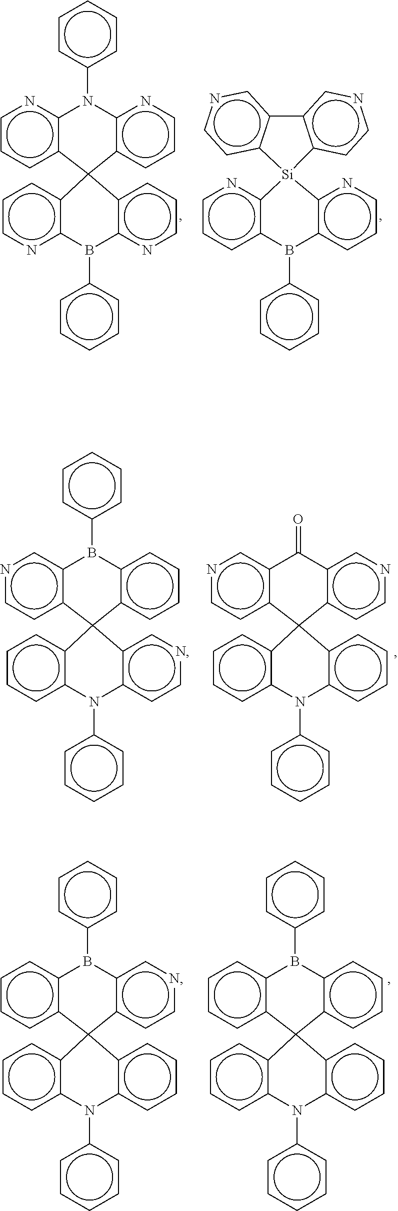

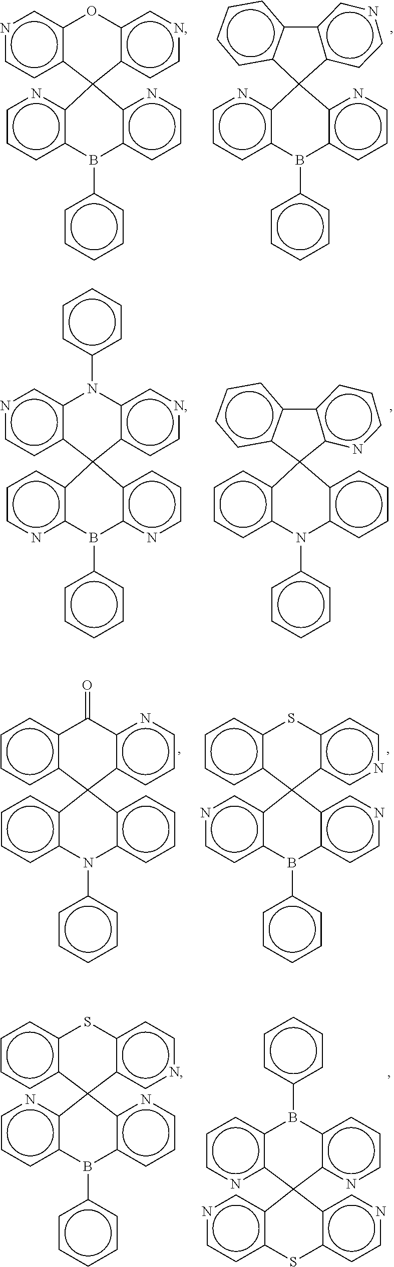

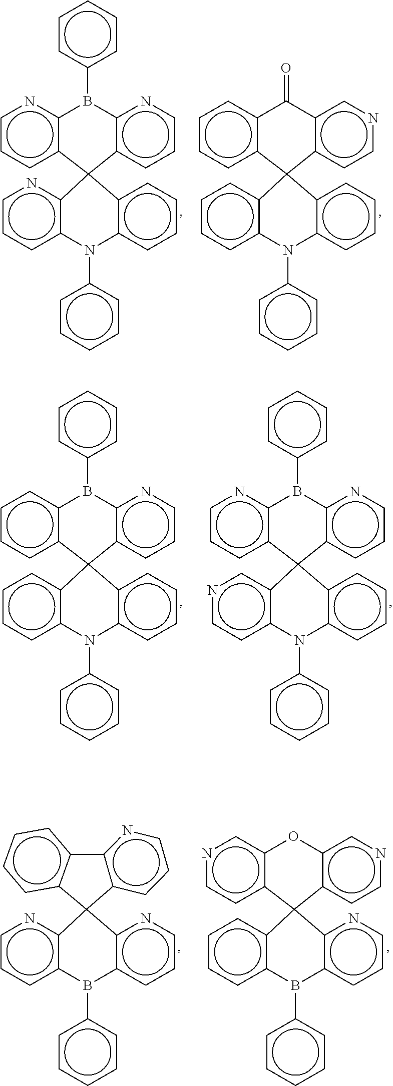

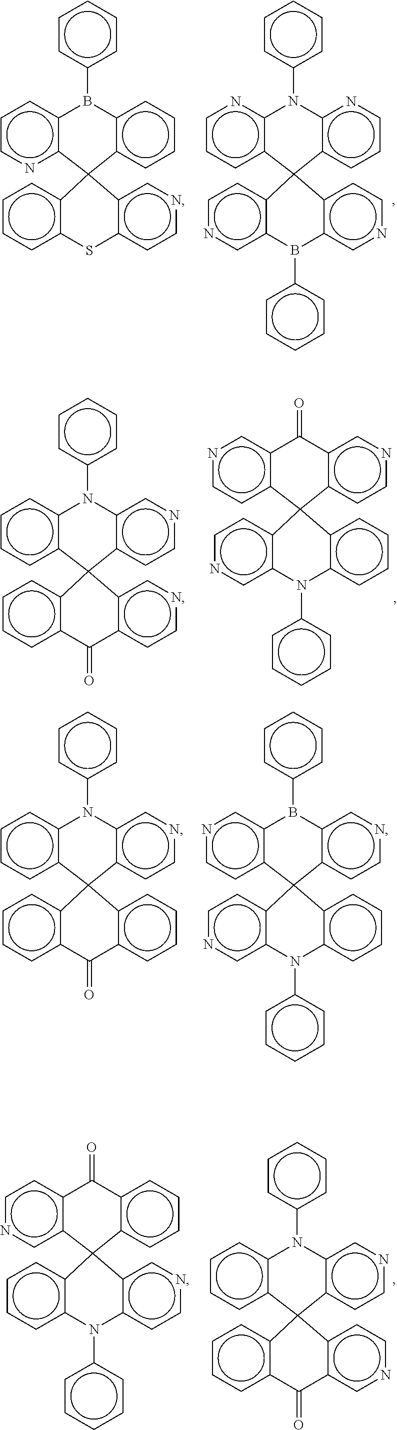

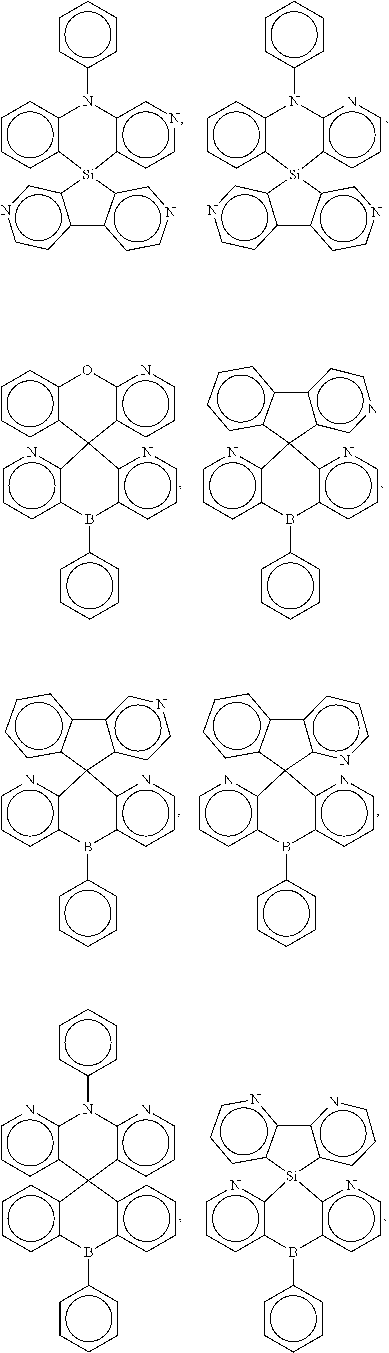

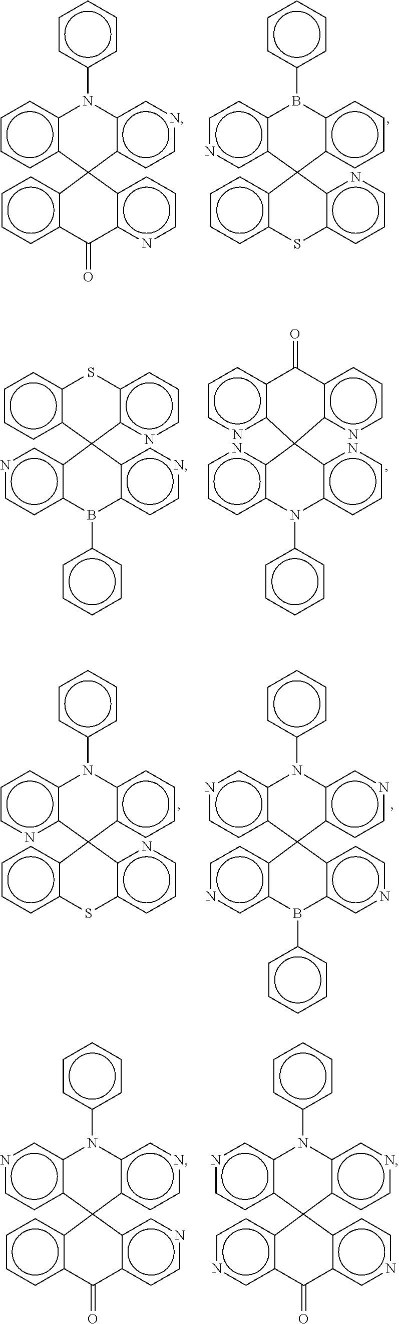

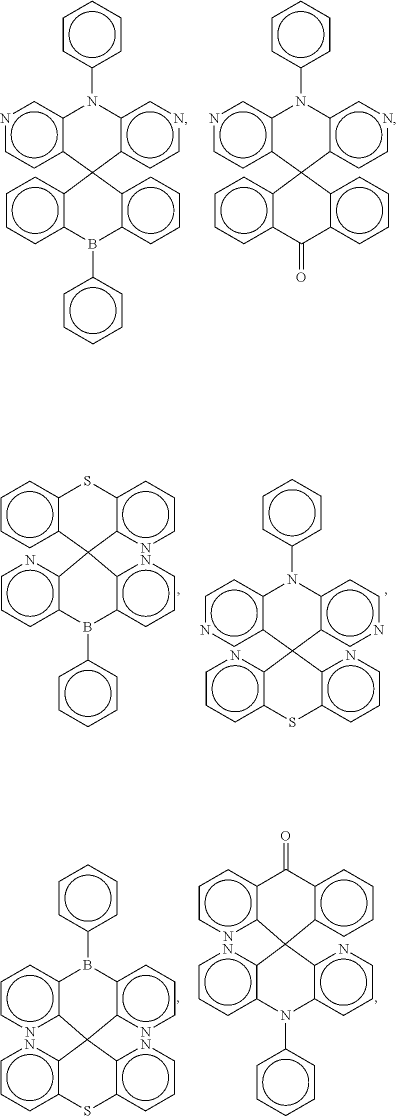

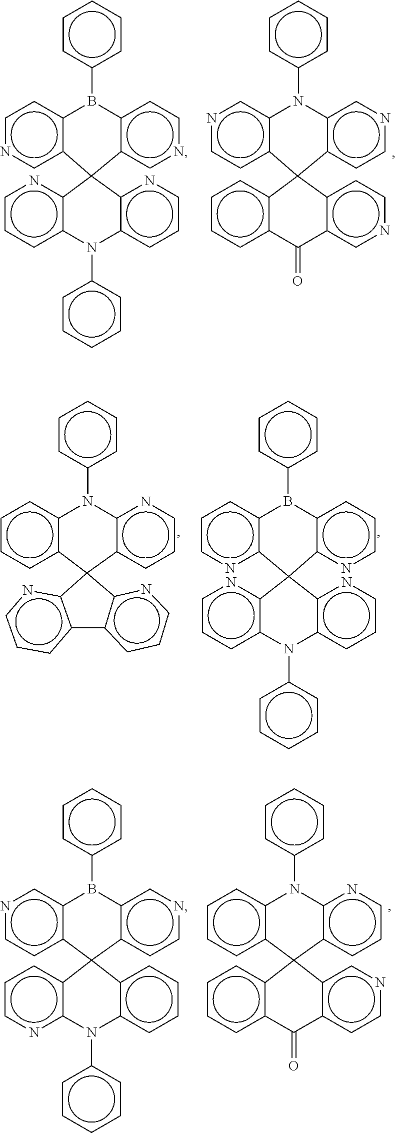

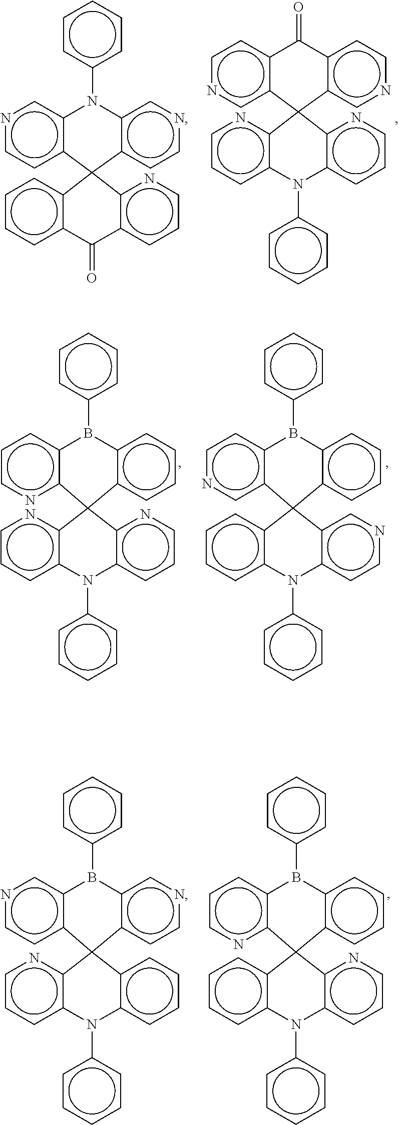

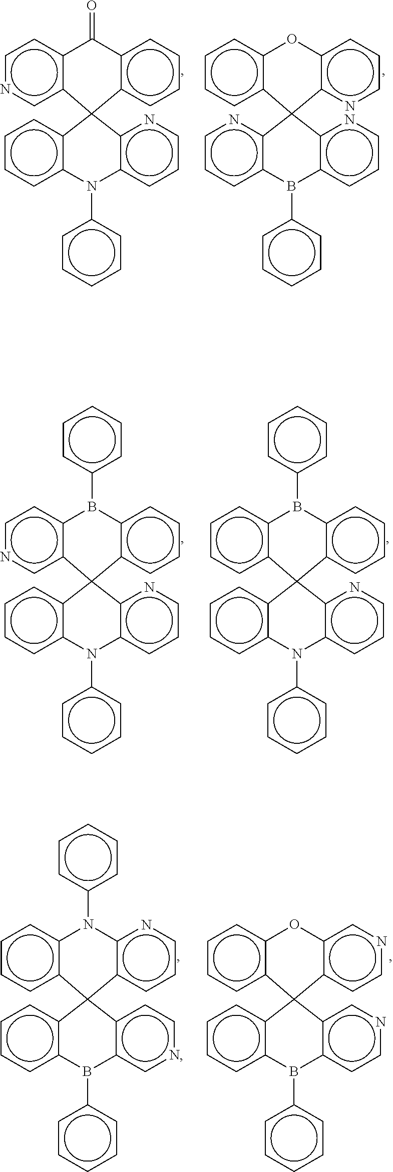

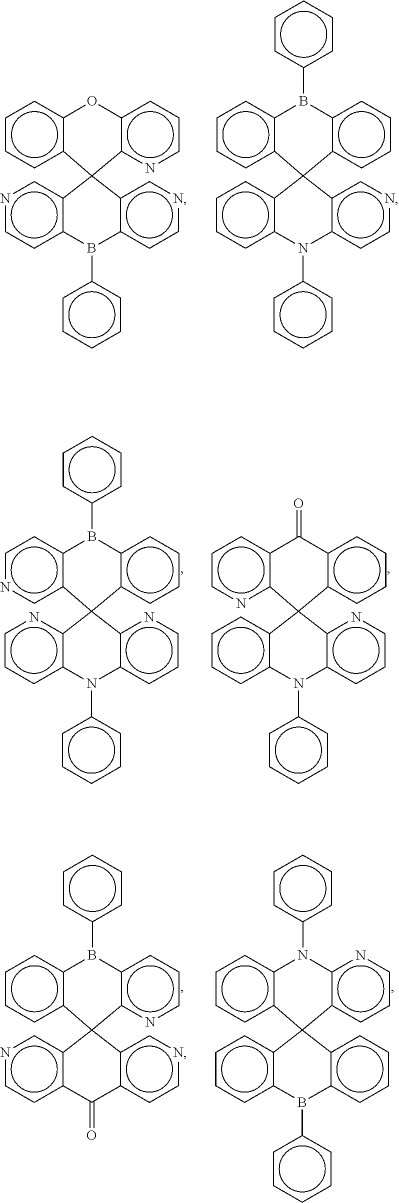

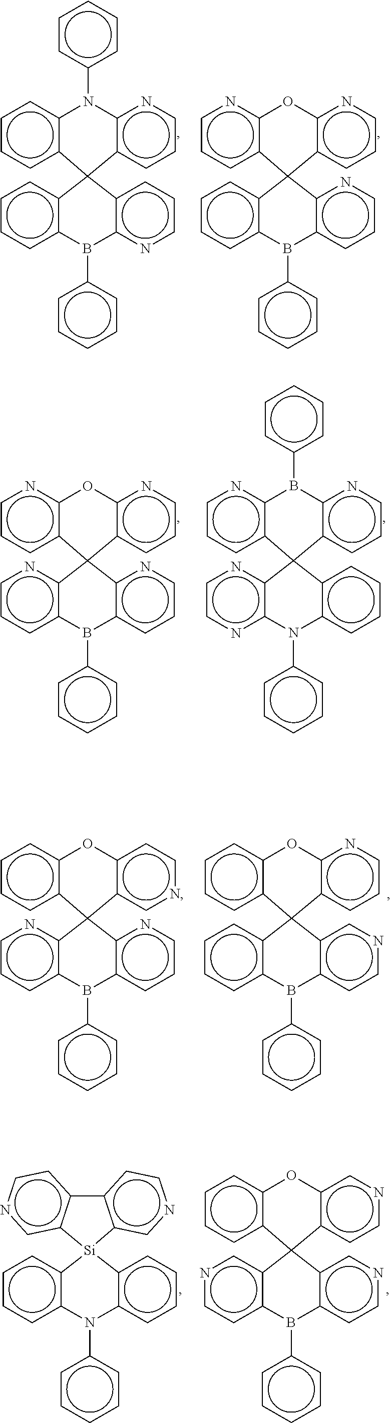

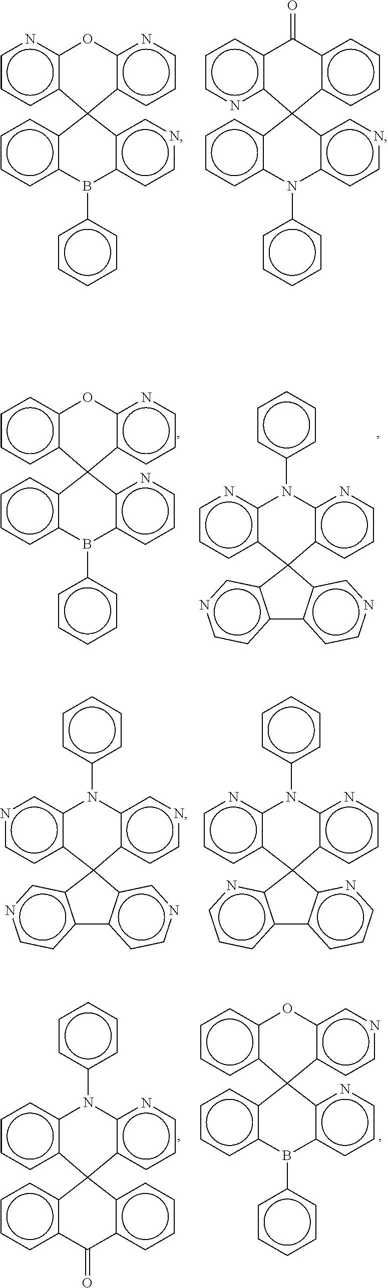

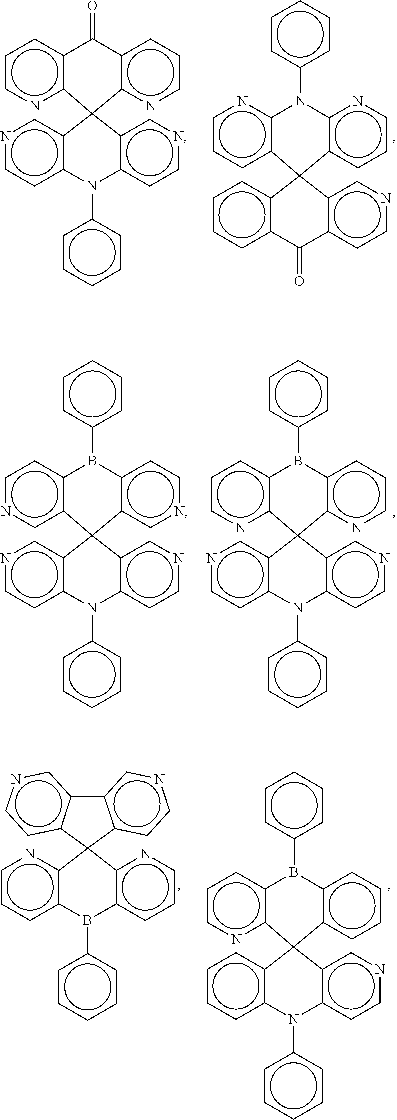

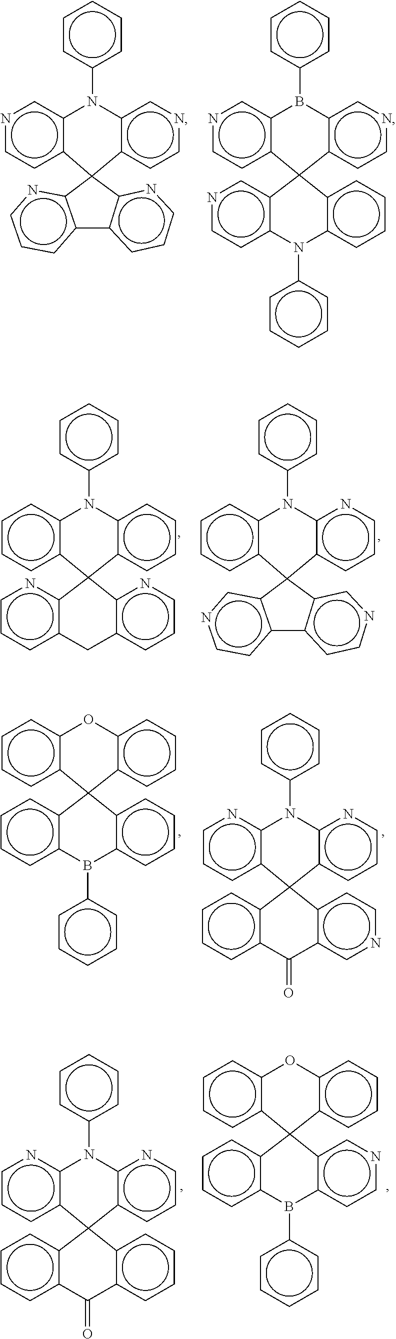

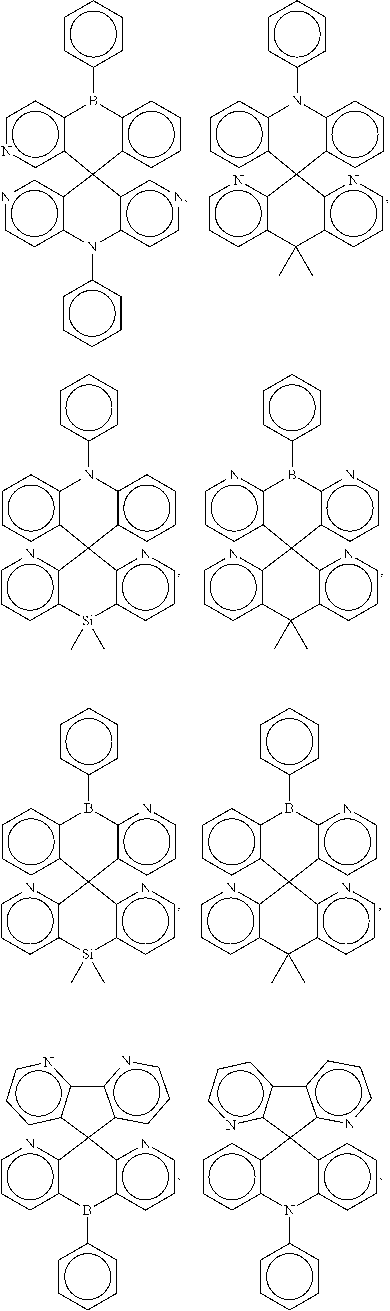

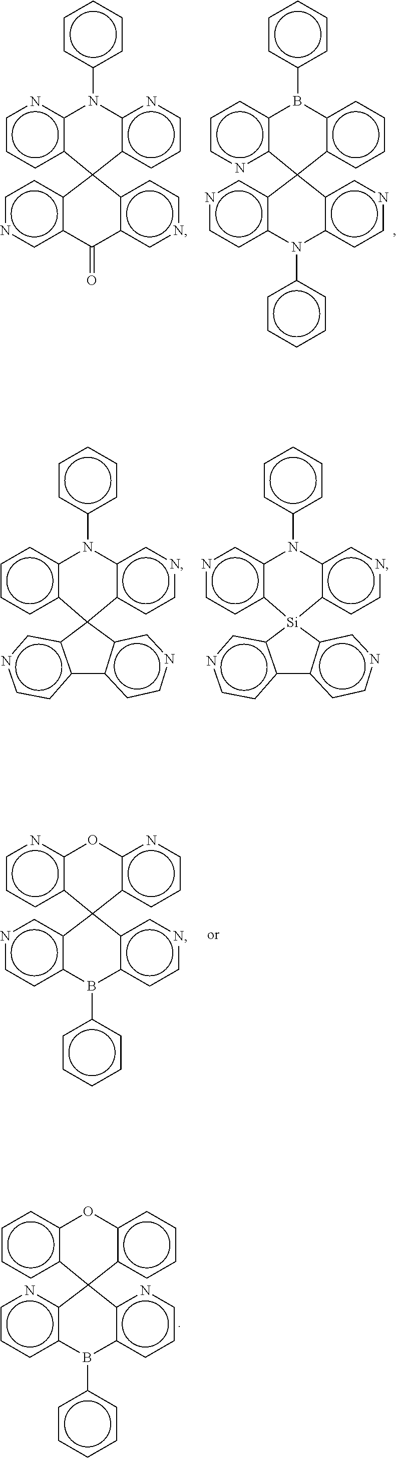

In some embodiments, the compound is a molecule represented by one of the above structural formulas, wherein any substitutable carbon is optionally substituted with R^{d}, and each R^{d} is independently selected from H, a C₁-C₆ alkyl, a C₃-C₁₈ cycloalkyl, a C₆-C₁₈ aryl, a 5-20 atom heteroaryl, halo, or —CN.

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIGS. 1 to 33 represent Table 1 which lists example embodiments of the present invention.

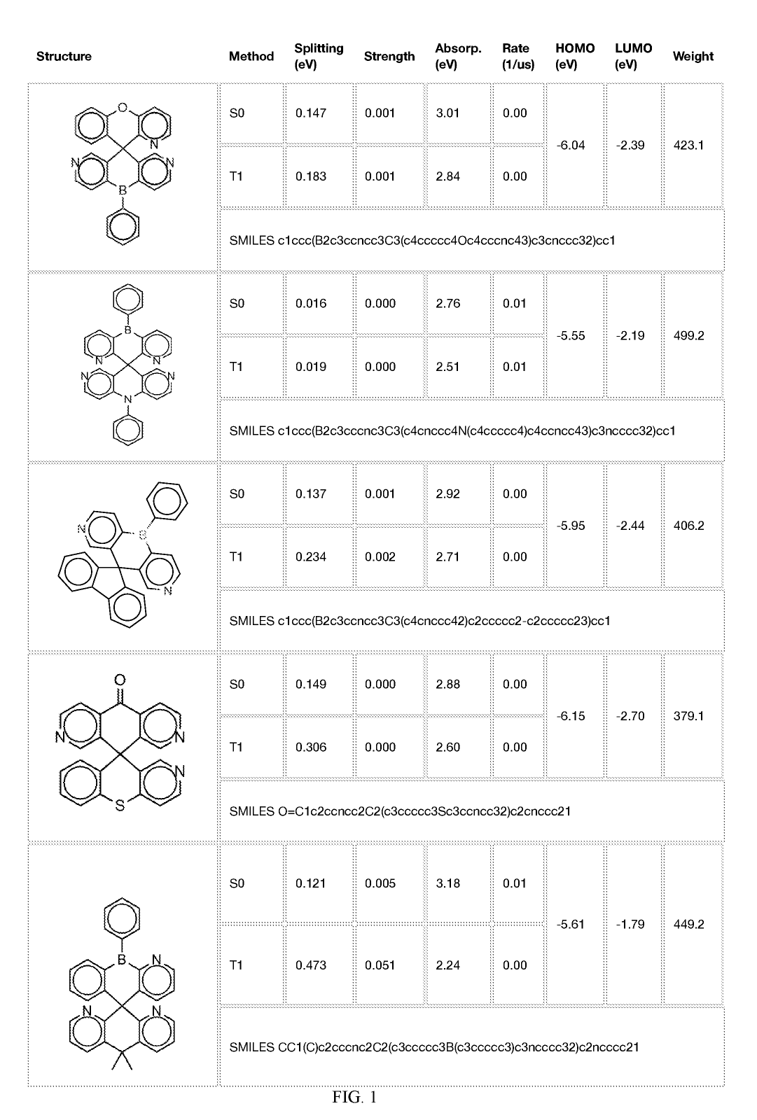

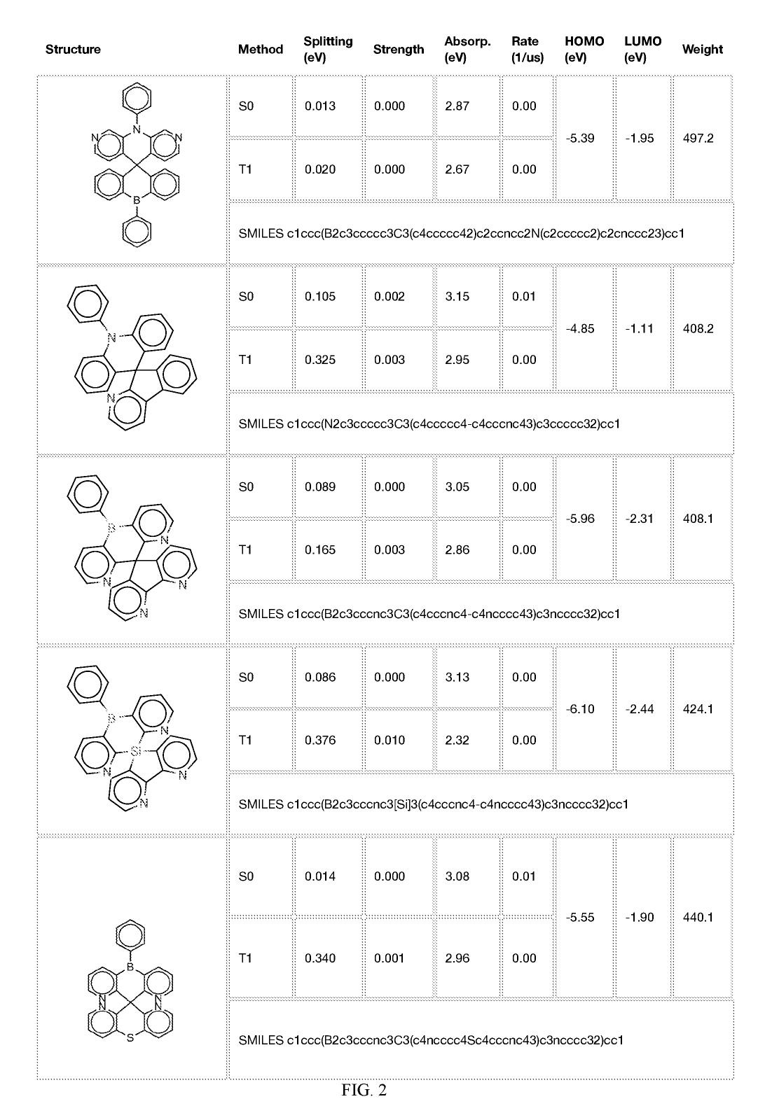

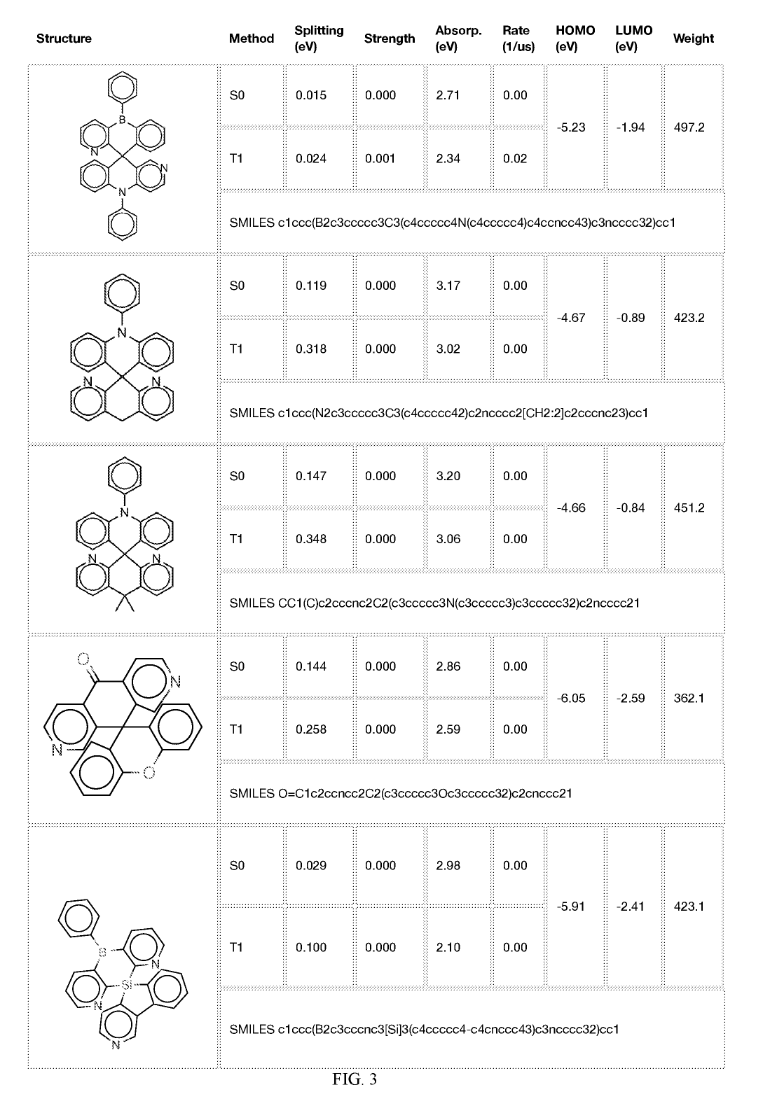

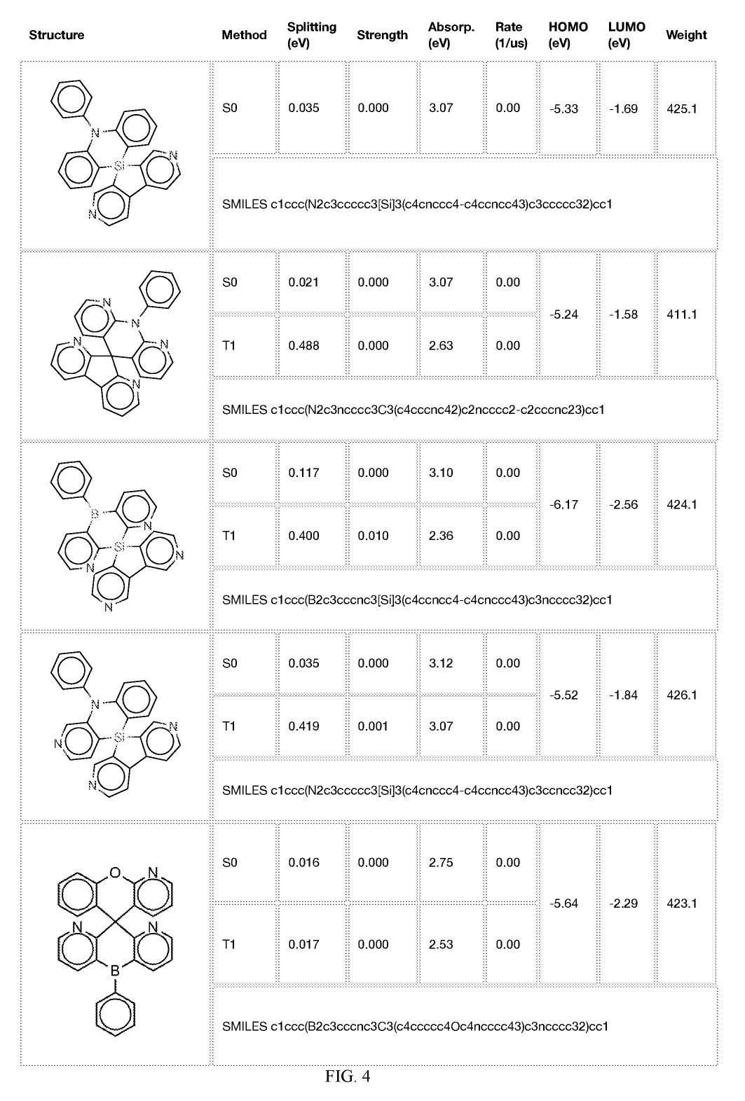

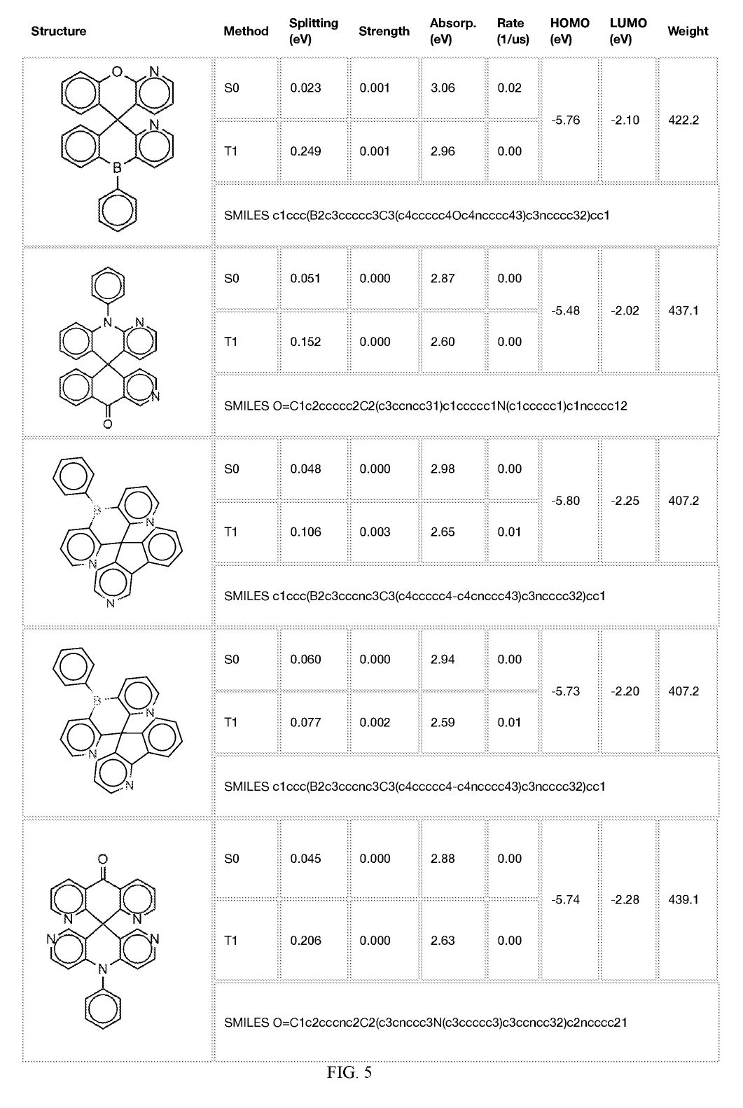

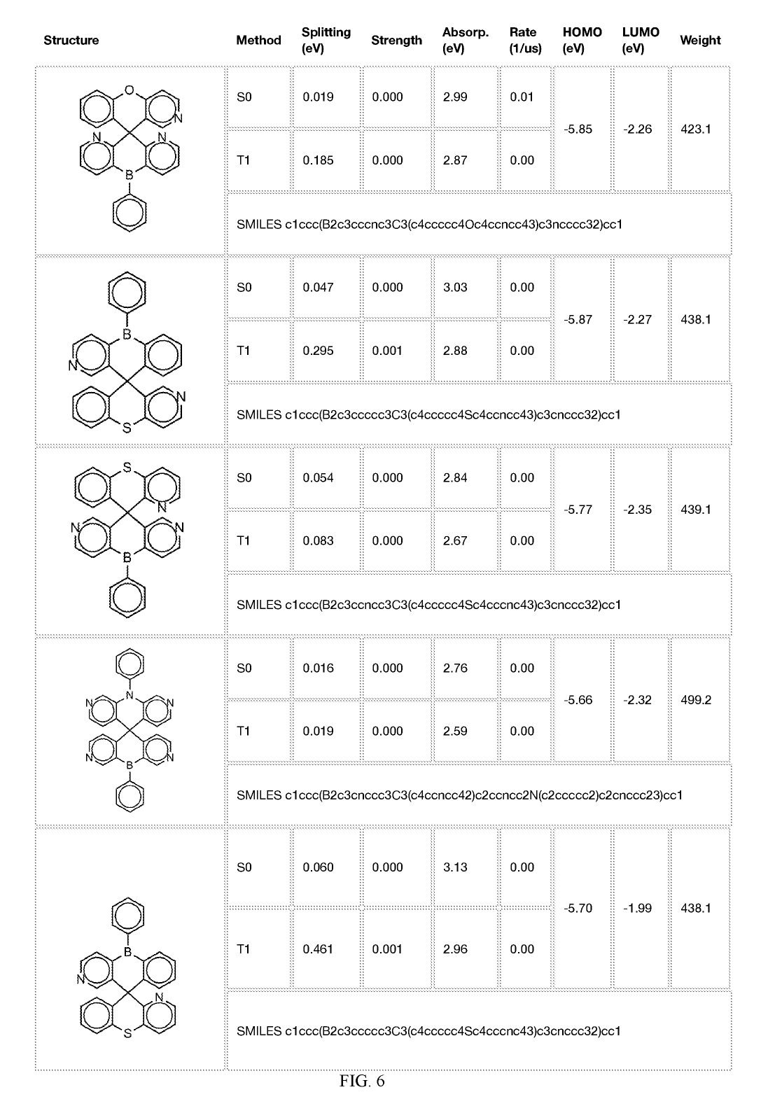

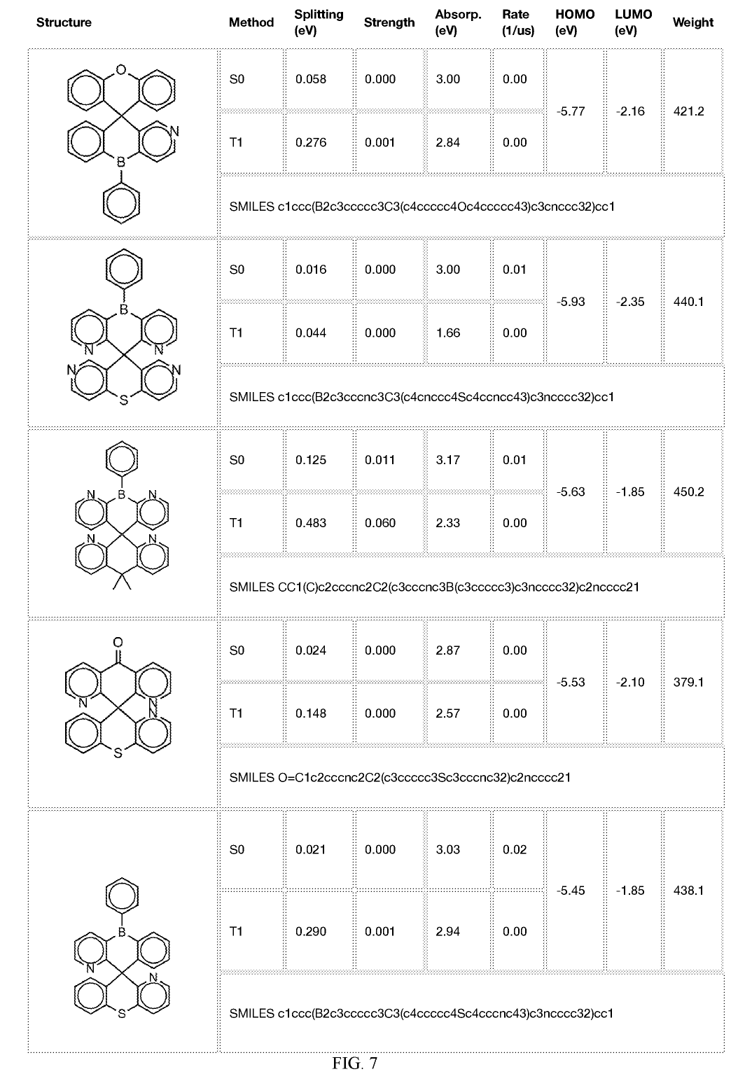

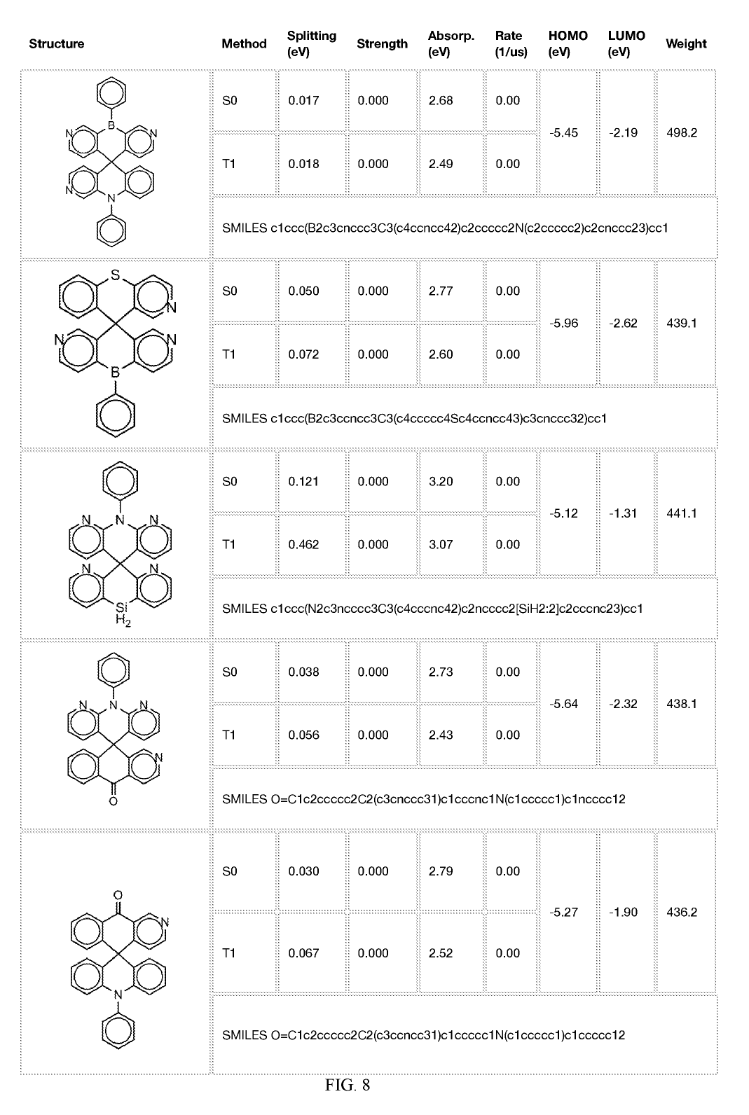

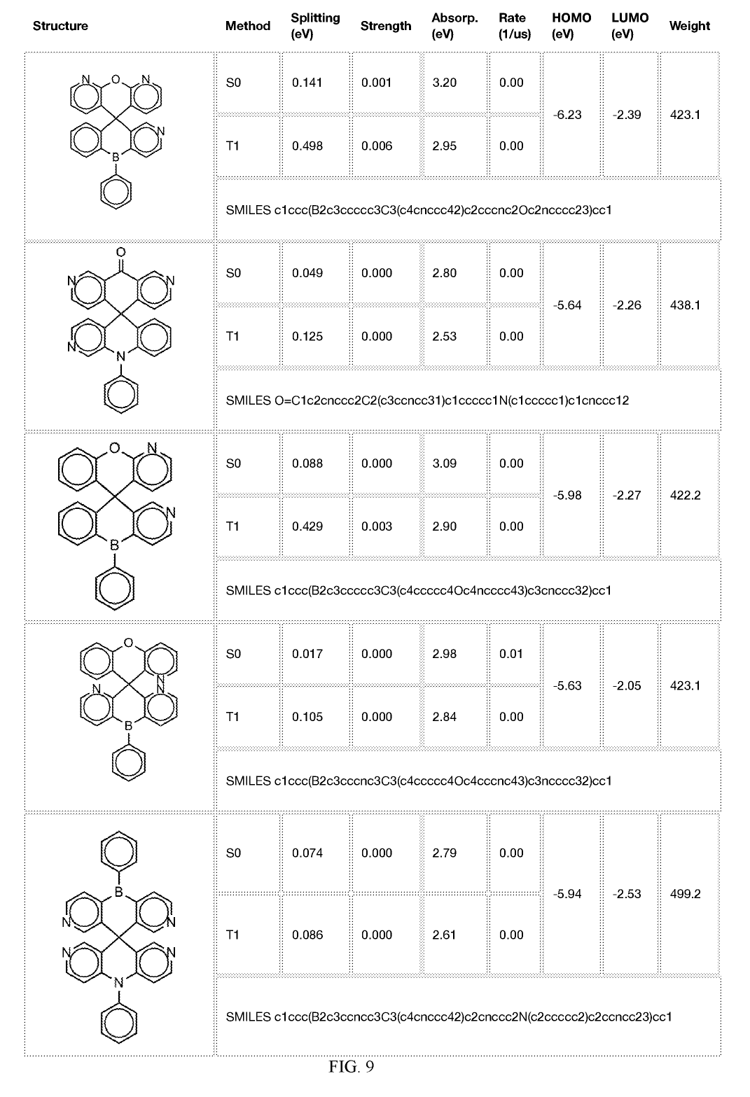

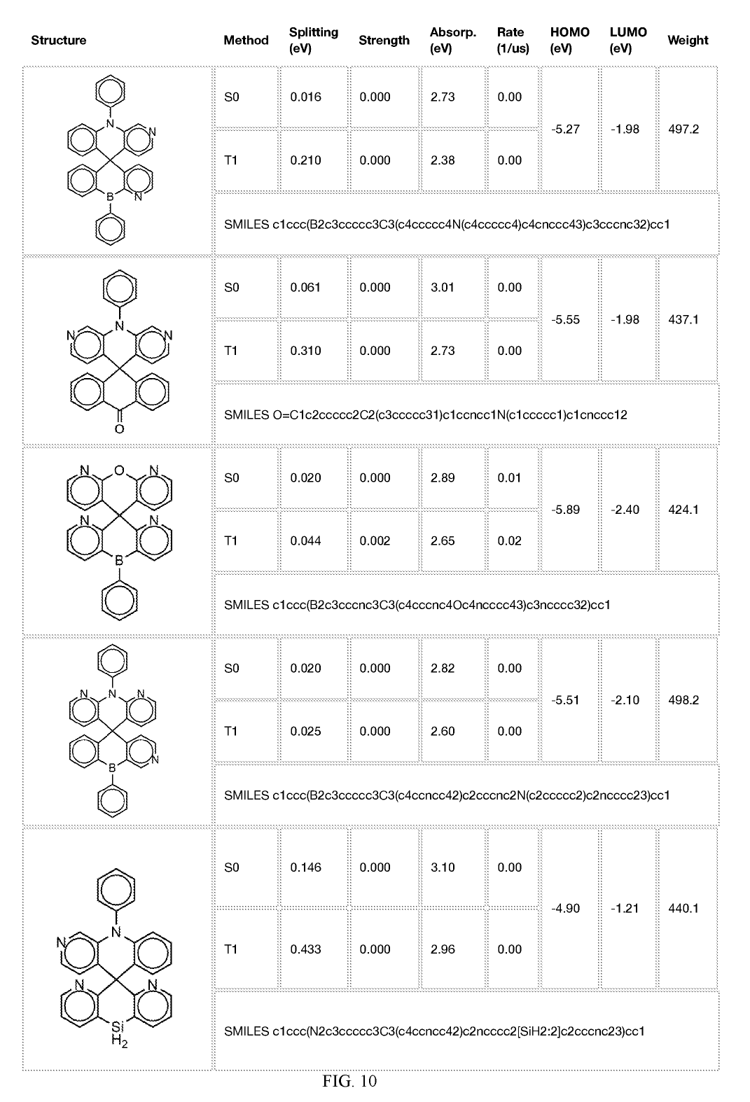

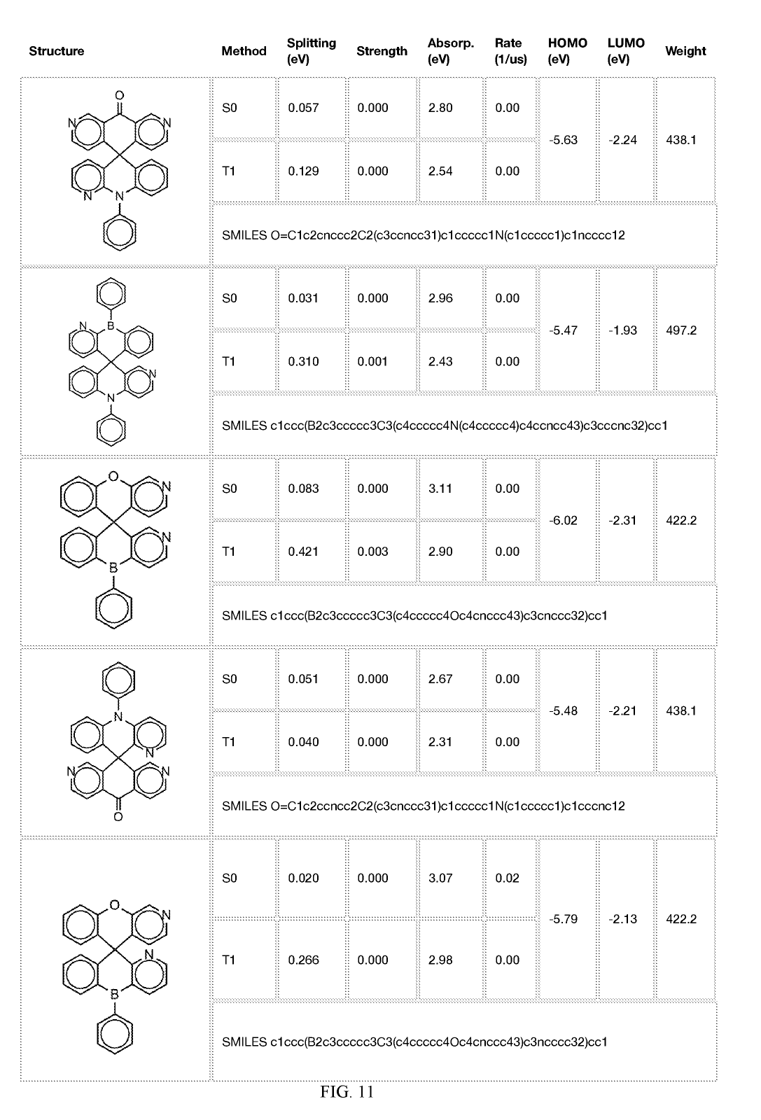

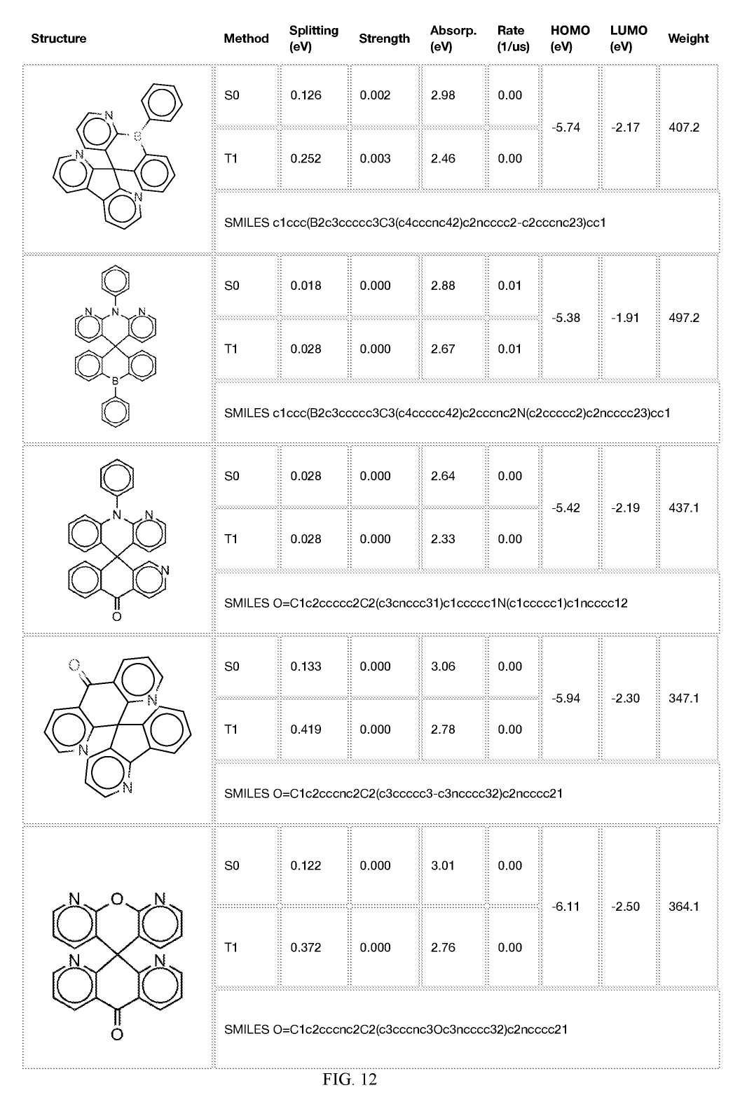

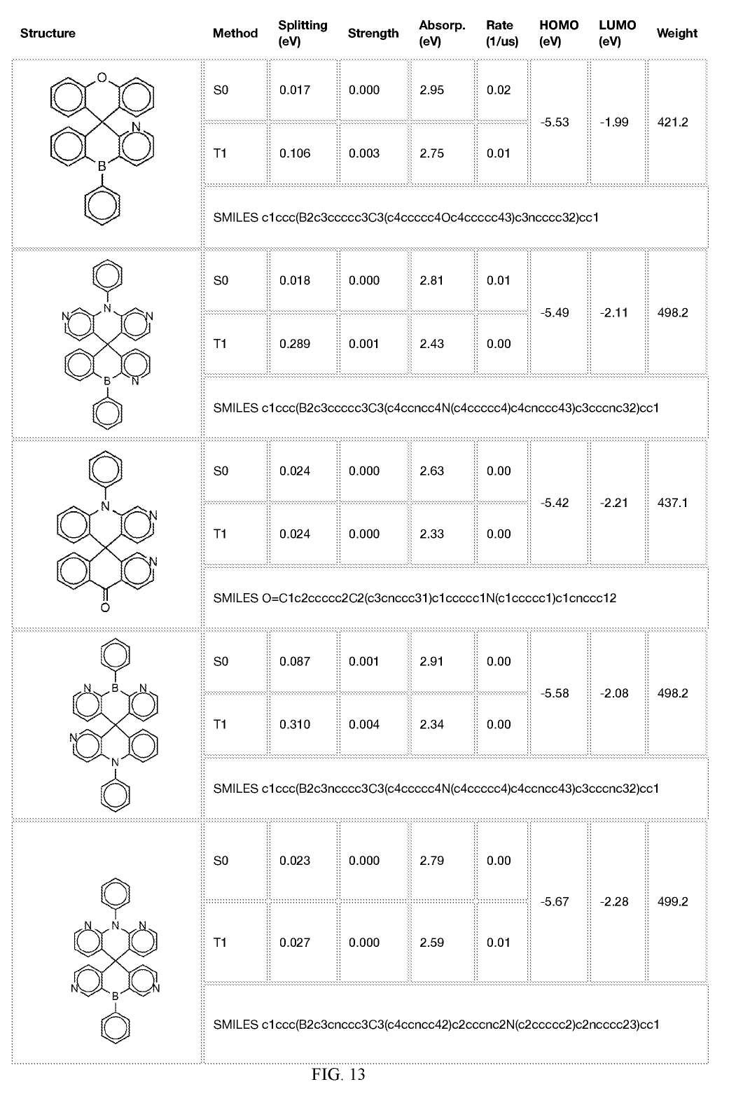

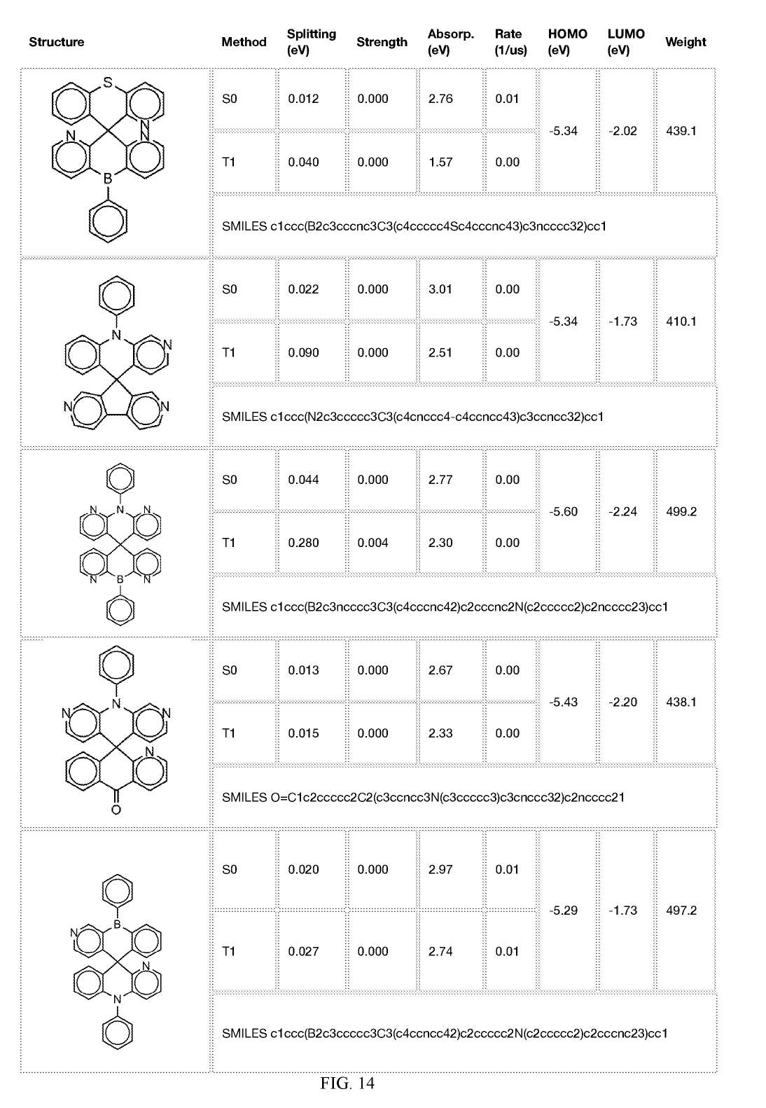

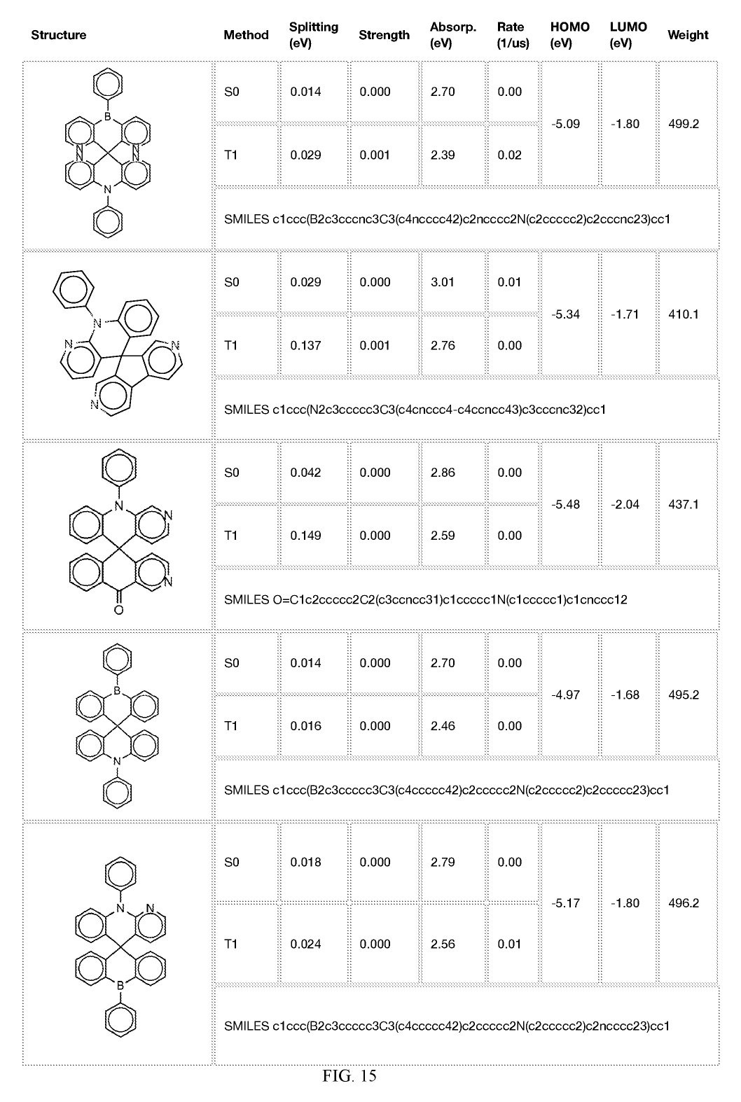

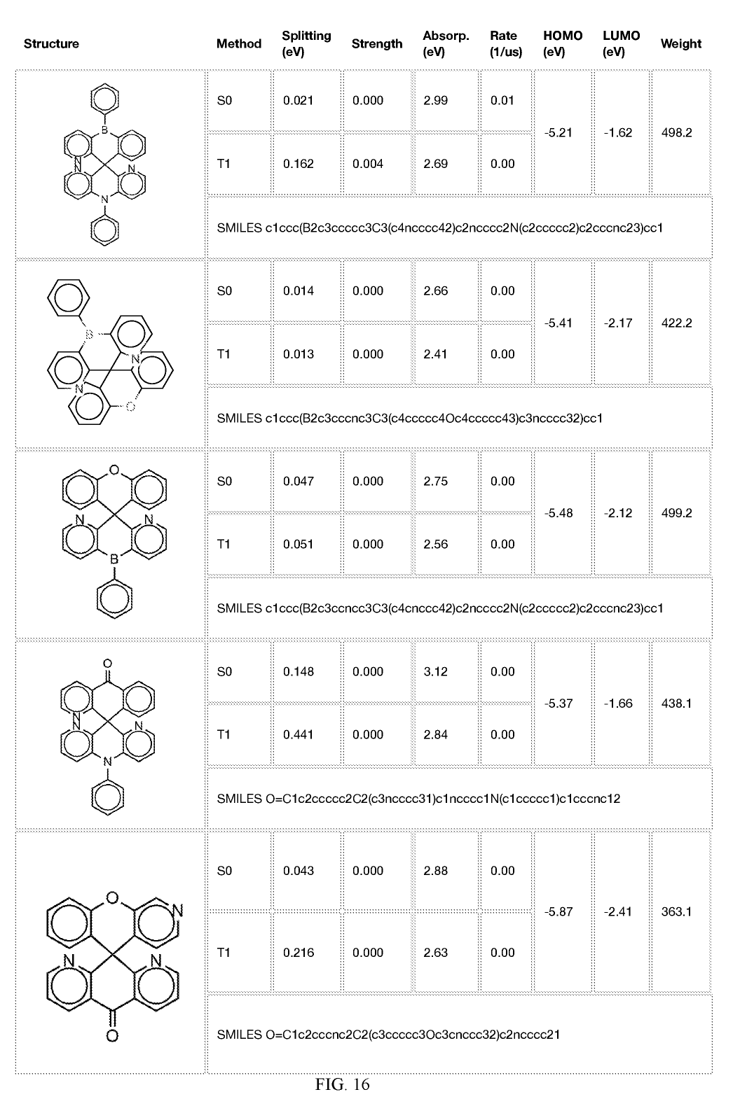

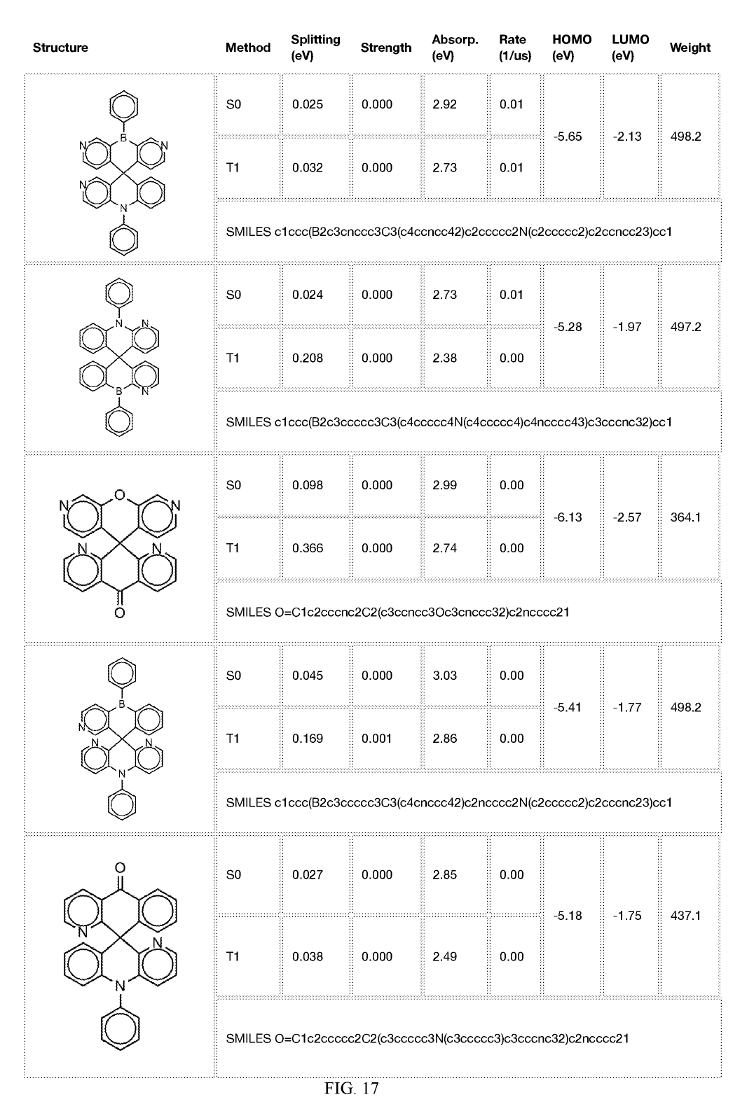

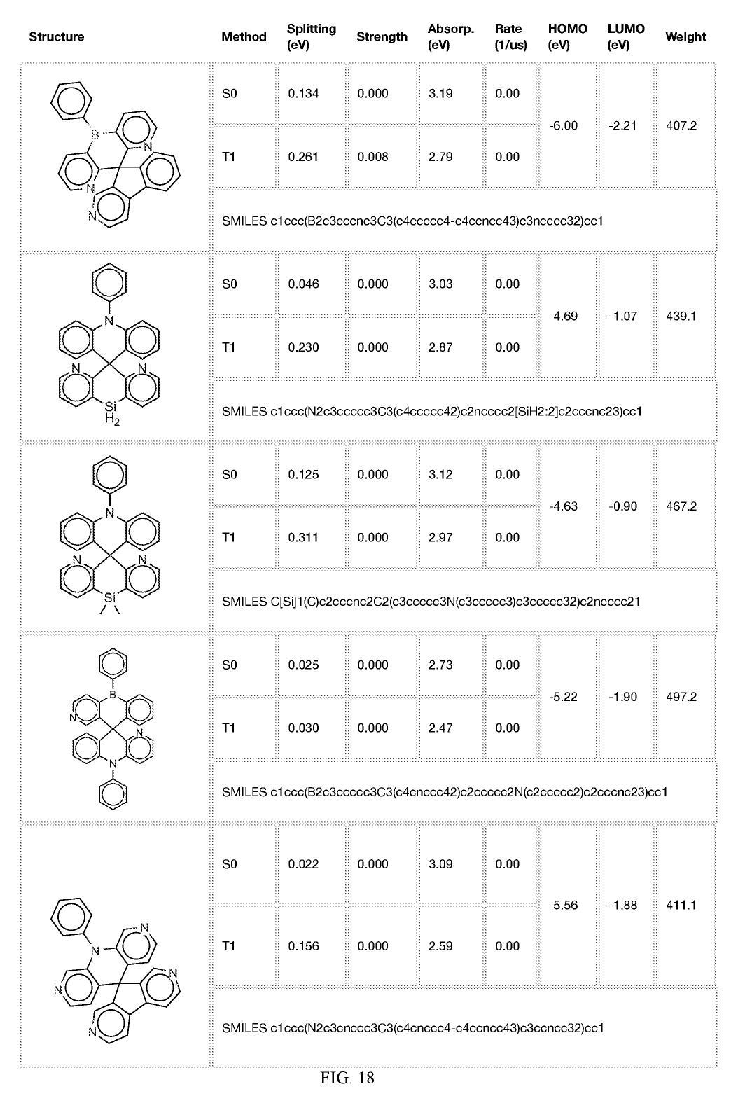

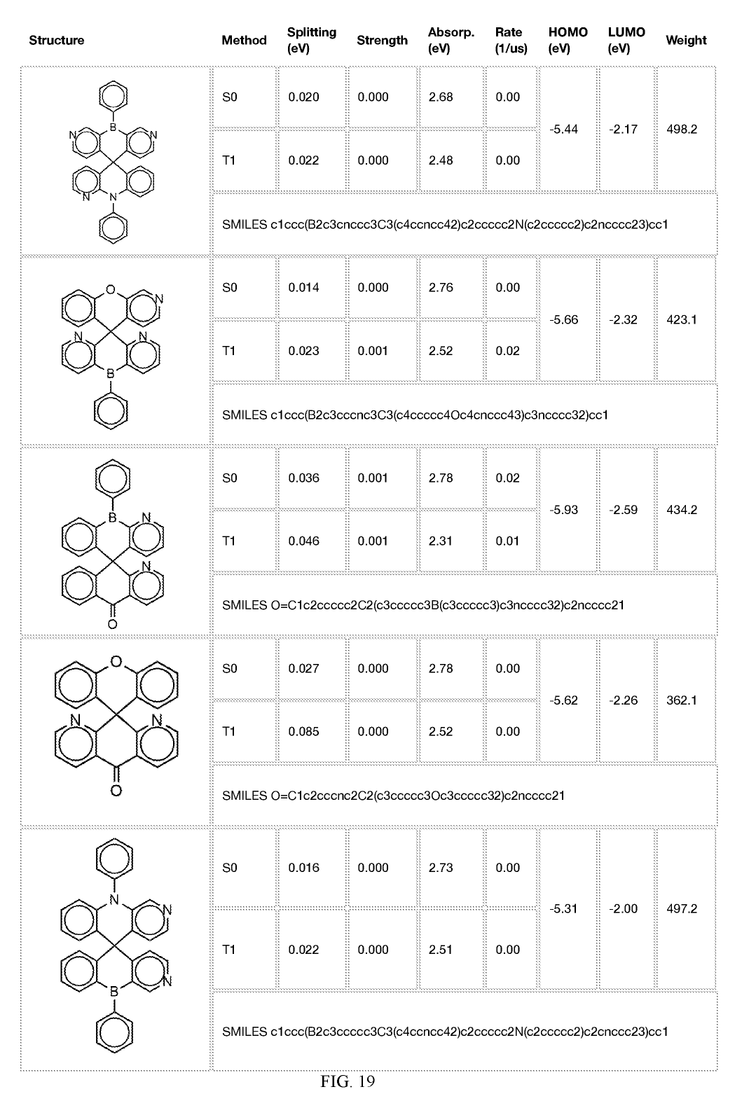

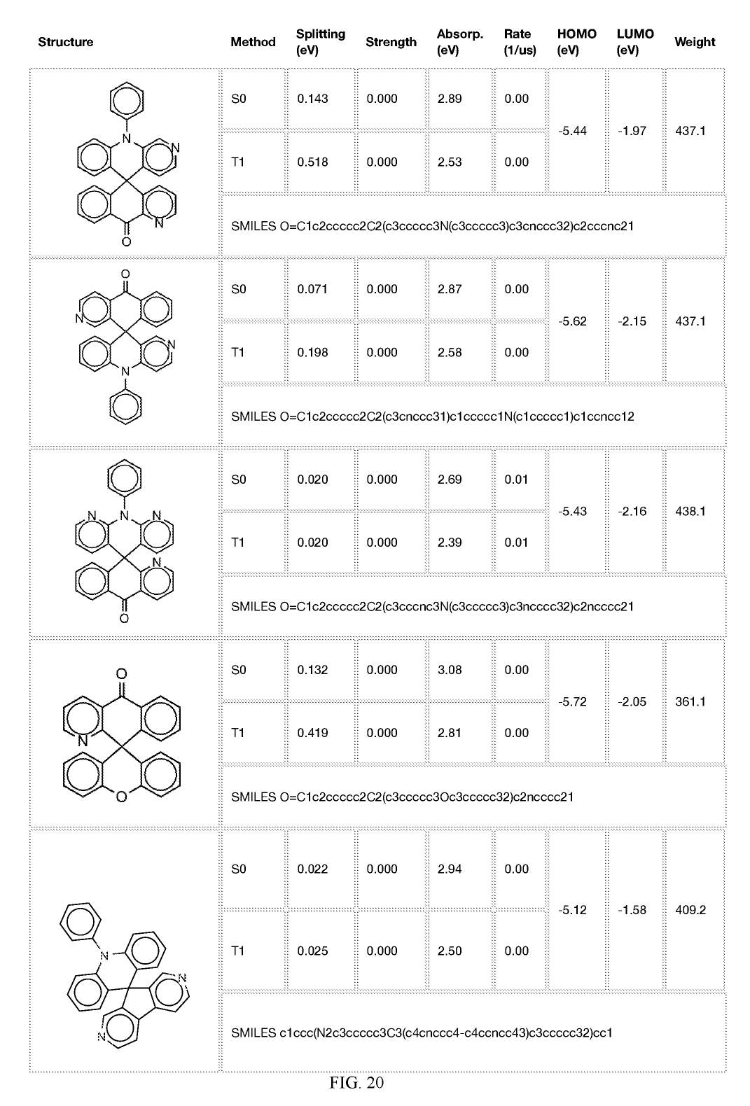

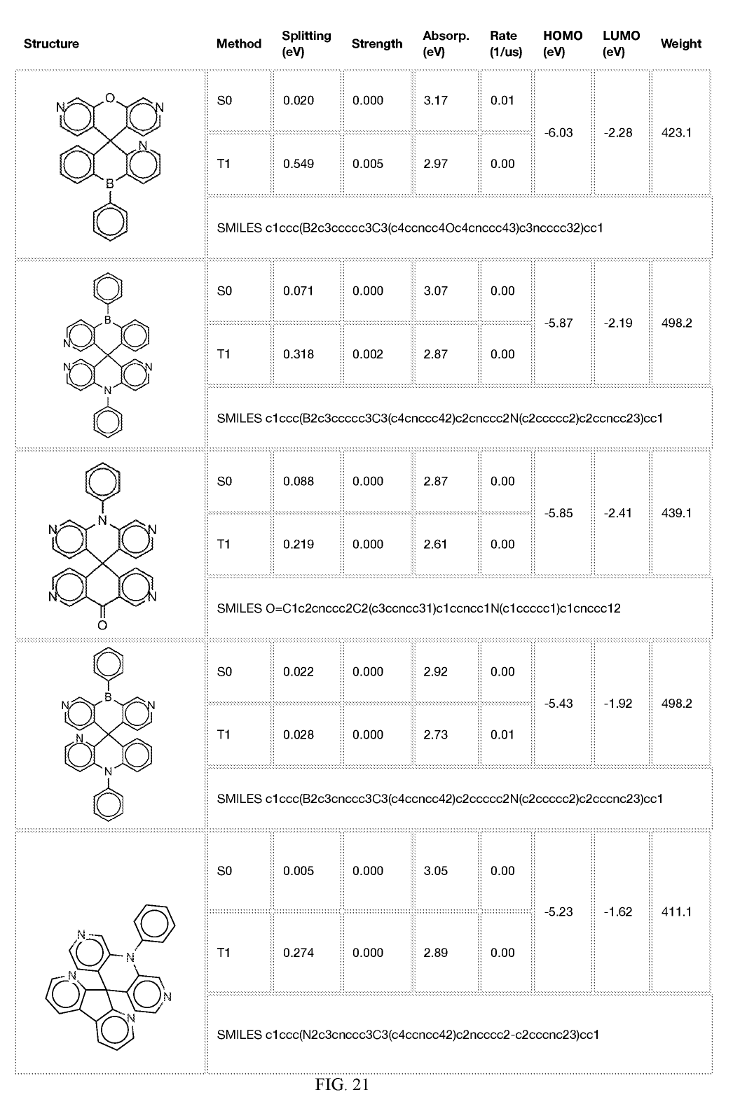

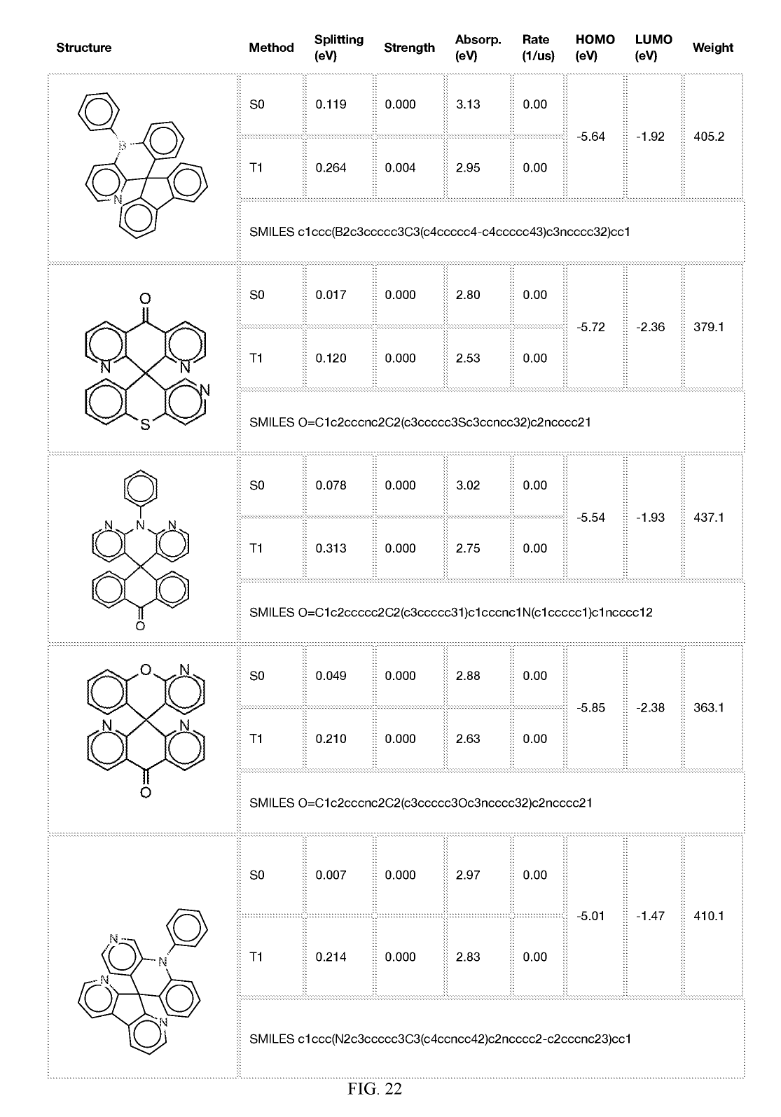

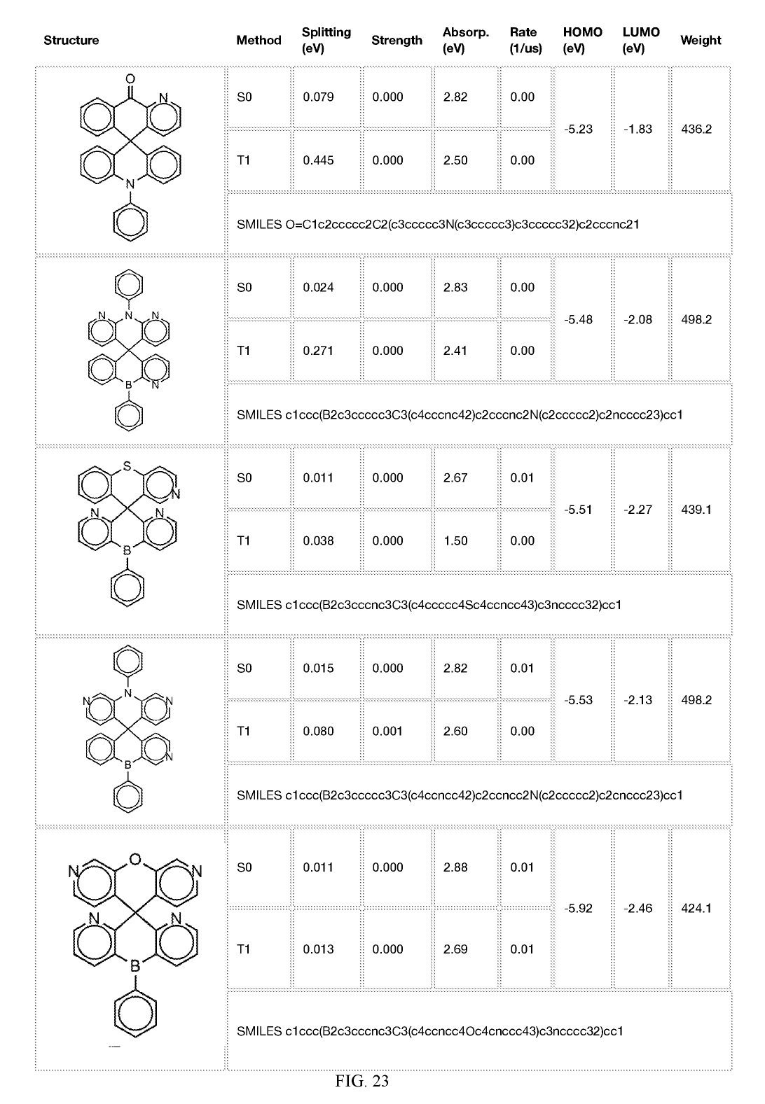

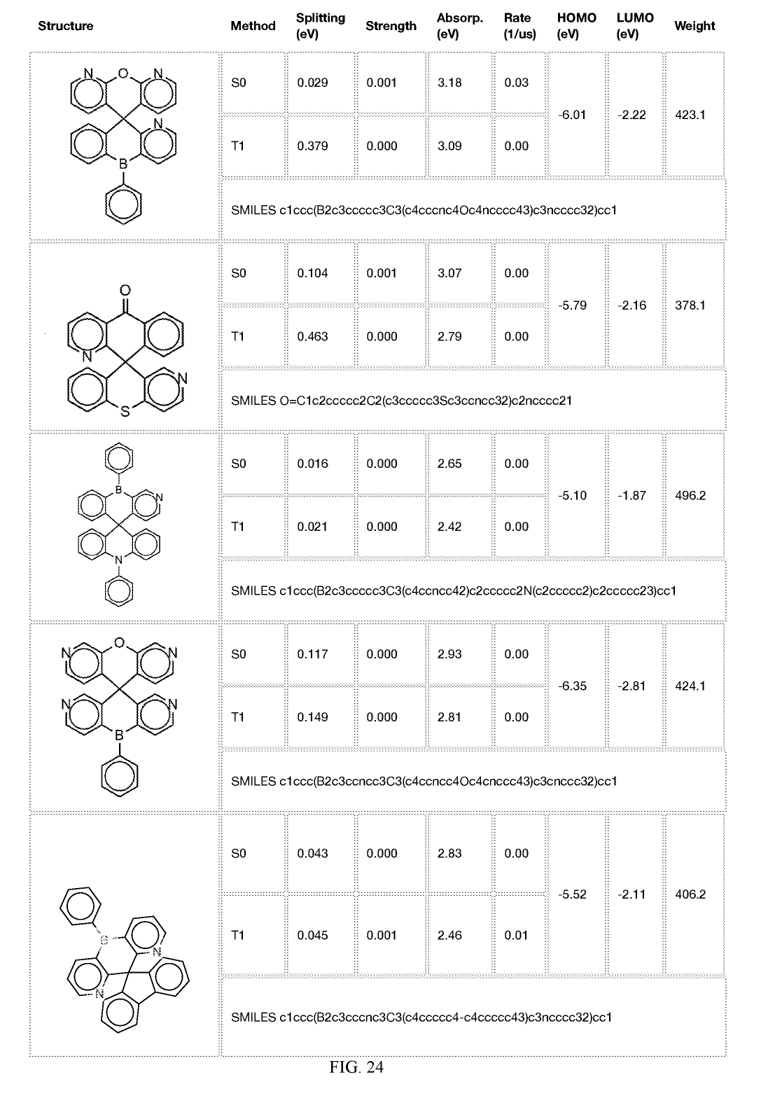

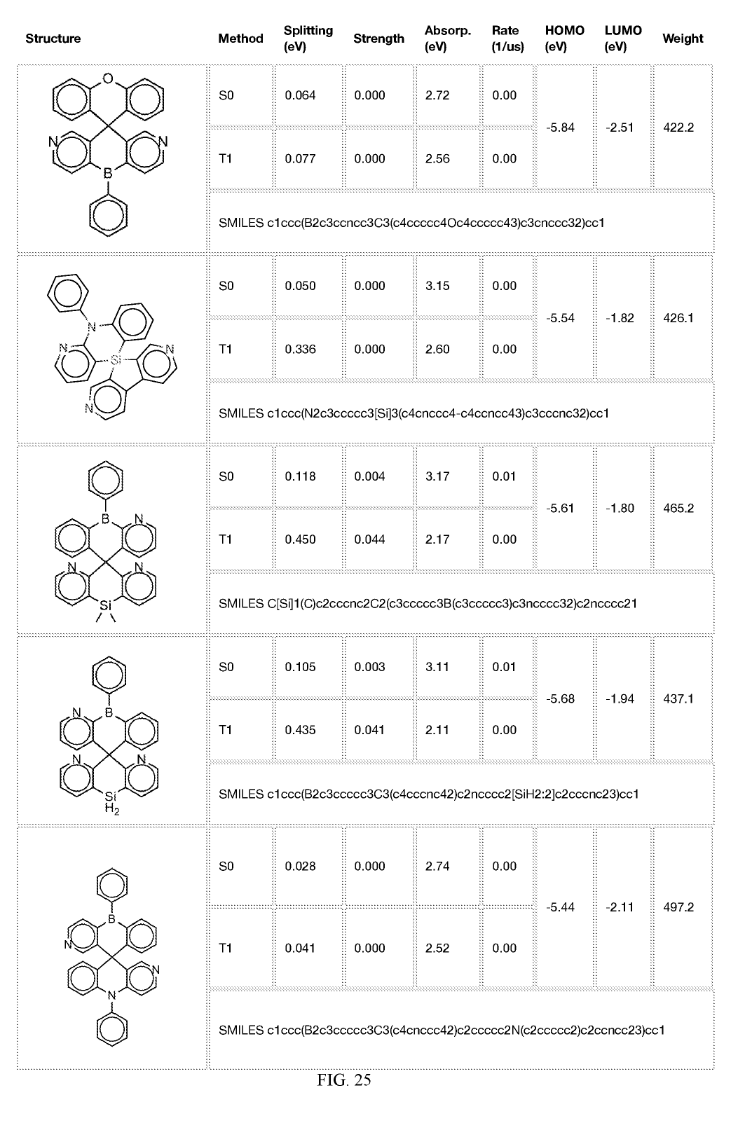

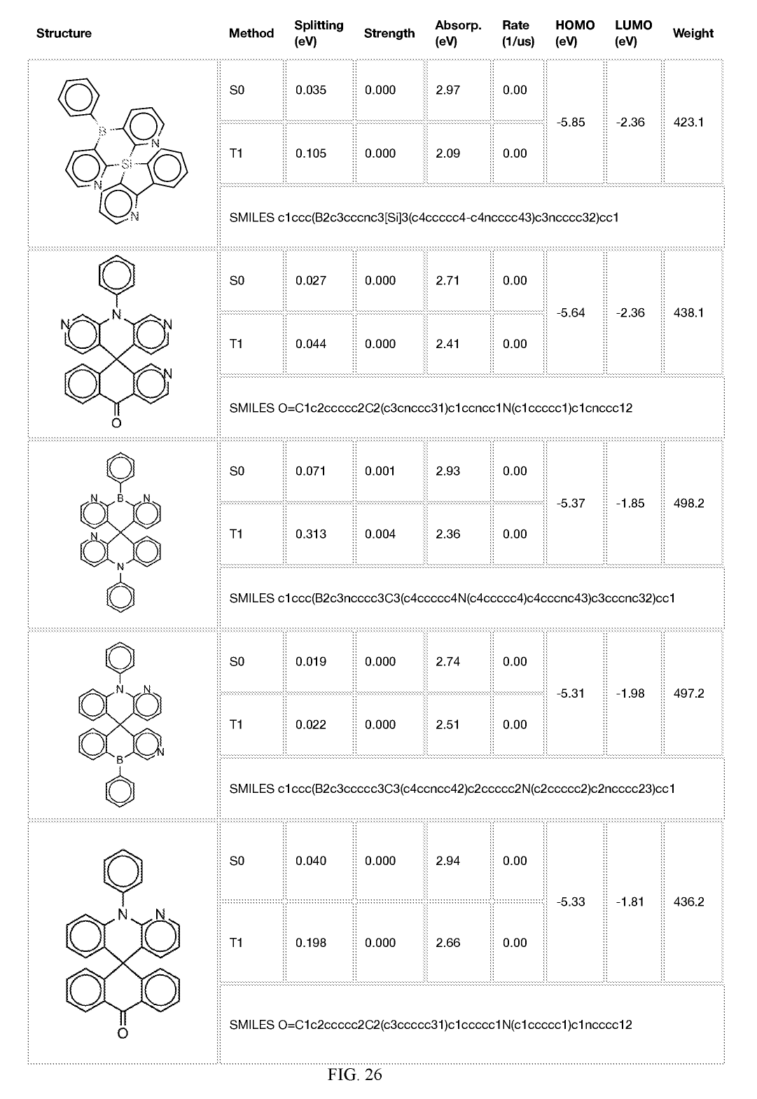

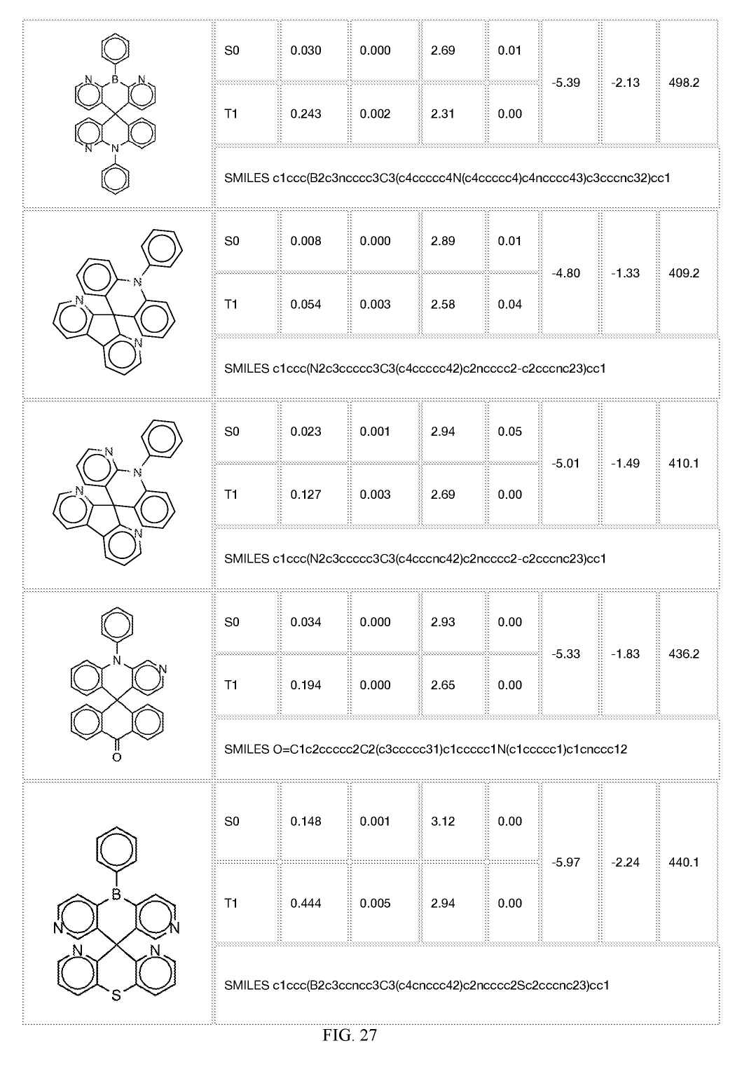

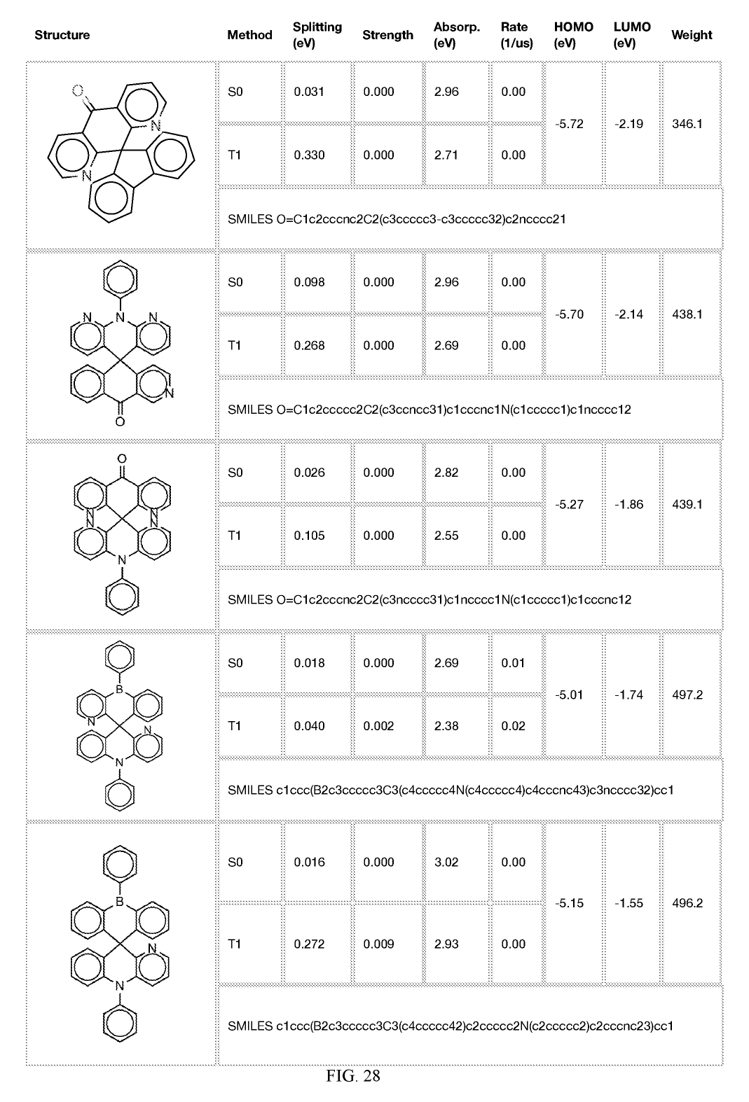

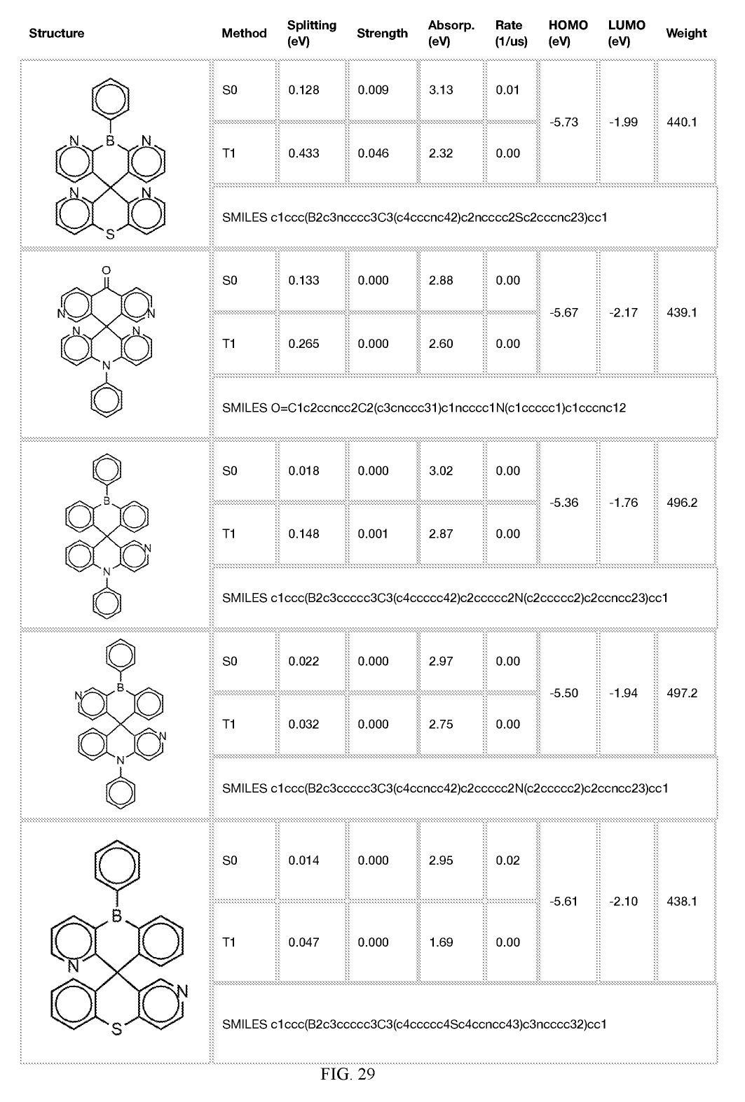

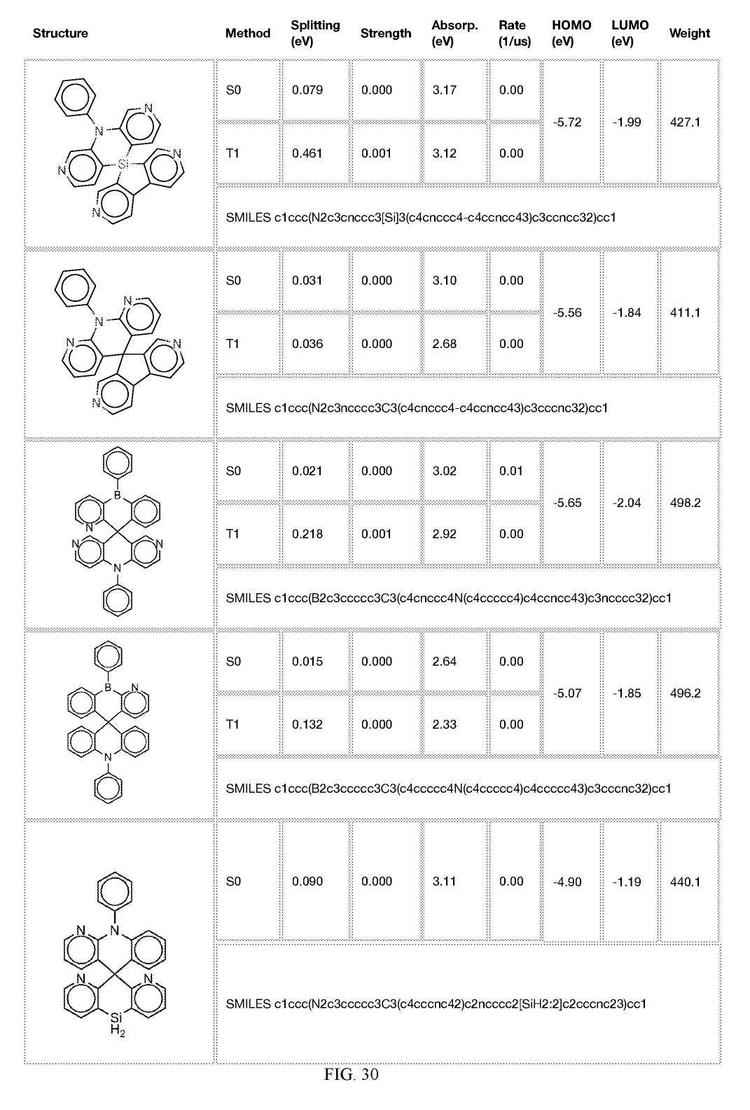

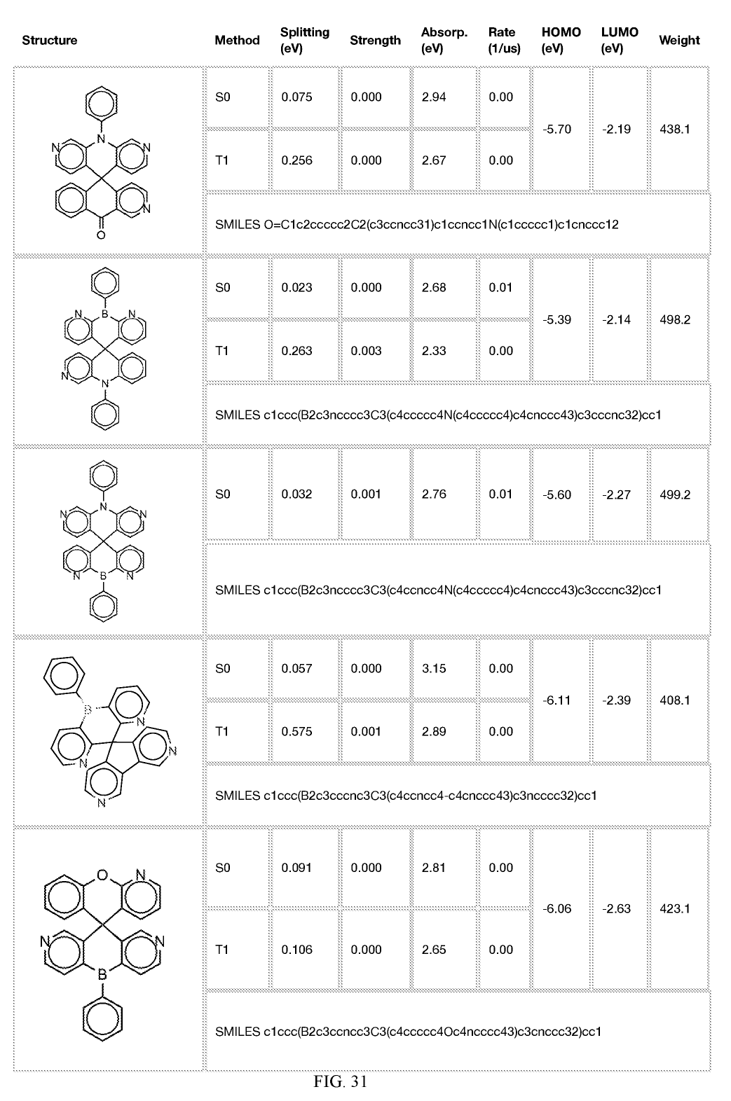

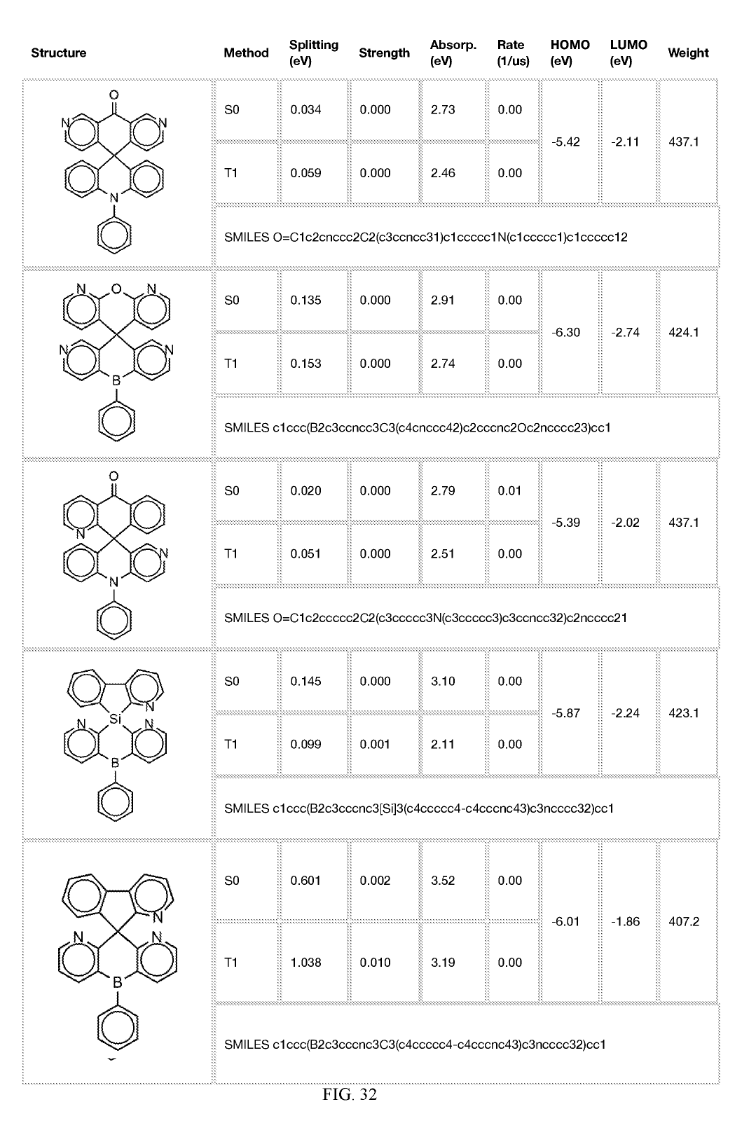

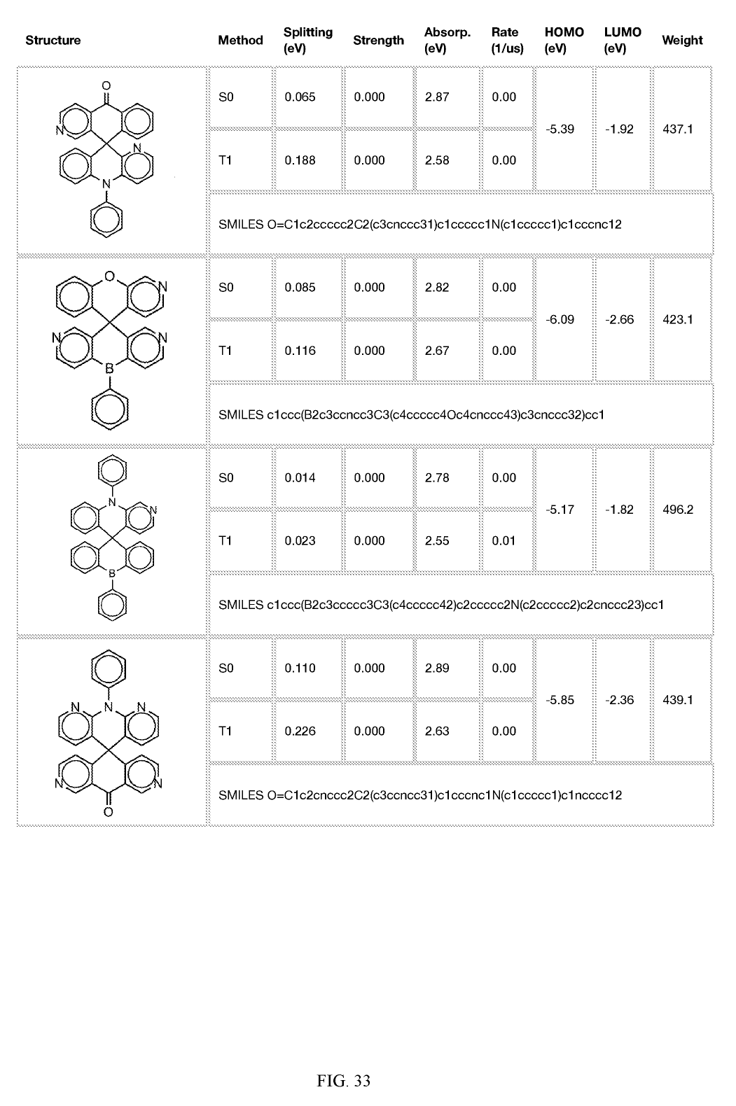

### DETAILED DESCRIPTION OF THE INVENTION

A description of example embodiments of the invention follows.

In some embodiments, the present invention relates to chemical molecules that are represented by a combination of fragments α and β, connected by a carbon atom:

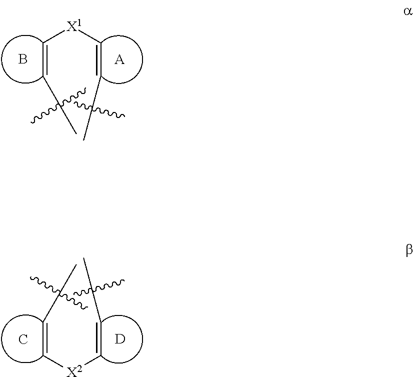

In example embodiments, fragments α and β are not identical. In other examples, fragments α and β are the same.

In various example embodiments, described below, fragments α and β can be the same or different.

For example, the molecules of the invention are represented by the following structural formula:

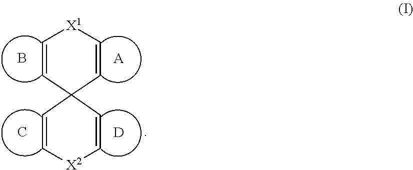

In structural formula (I) of the present invention:
X¹ and X² are independently selected from O, S, C(O), CR^{a}R^{b}. SiR^{a}R^{b}, NR^{c}, BR^{c}, or a bond. Each of A, B, C, and D is, independently, an optionally substituted six-membered aromatic or heteroaromatic ring, wherein at least one of rings A, B, C, and D contains at least one Nitrogen atom.
Each instance of R^{a}, R^{b}, and R^{c} is independently selected from H, a C₁-C₆ alkyl, a C₃-C₁₈ cycloalkyl, a C₆-C₁₈ aryl, a 5-20 atom heteroaryl, halo, or —CN.

In some embodiments at least one of X₁ and X² is not CH₂.

In some embodiments, the molecule of formula (1) is not group symmetric. In some embodiments, the molecule of formula (I) is group symmetric.

In some embodiments, ring A, B, C, or D is substituted with one or more substituents selected from a C₁-C₆ alkyl, a C₃-C₁₈ cycloalkyl, a C₆-C₁₈ aryl, a 5-20 atom heteroaryl, halo, or —CN.

In some embodiments, at least one of rings A, B, C, and D contains at least one N. In some embodiments, at least two of rings A, B, C, and D contain at least one N. In some embodiments, at least three of rings A, B, C, and D contain at least one N. In some embodiments, each of rings A, B, C, and D contains at least one N.

In some embodiments, at least one of rings A, B, C, and D is a six-membered ring. In some embodiments, at least two of rings A, B, C, and D are six-membered rings. In some embodiments, at least three of rings A, B, C, and D are six-membered rings. In some embodiments, each of rings A, B, C, and D is a six-membered ring.

In some embodiments, each of rings A, B, C, and D contains 0, 1, or 2 Nitrogens. In some embodiments, each of rings A, B, C, and D contains 0 or 1 Nitrogen.

In some embodiments, the structure of formula (I) can be represented by the following structural formula:

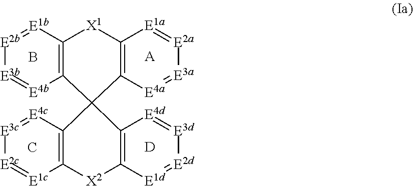

In the structure of formula (Ia):
Each atom E^{1a}, E^{2a}, E^{3a}, E^{4a}, E^{1b}, E^{2b}, E^{3b}, E^{4b}, E^{1c}, E^{2c}, E^{3c}, E^{4c}, E^{1d}, E^{2d}, E^{3d}, and E^{4d} is independently selected from CR^{d} or N.
Each instance of R^{d} is independently selected from H, a C₁-C₆ alkyl, a C₃-C₁₈ cycloalkyl, a C₆-C₁₈ aryl, a 5-20 atom heteroaryl, halo, or —CN.

In some embodiments, X¹ is SiR^{a}R^{b}, BR^{c}, O, or S. In some embodiments, X¹ is SiR^{a}R^{b} or BR^{c}.

In some embodiments, each instance of R^{a}, R^{b}, and R^{c} is independently selected from H, phenyl, and C₁₋₃ alkyl.

In some embodiments, each instance of R^{d} is H or C₁-C₃ alkyl.

In some embodiments, X₁ is B-phenyl and X₂ is O, S, N-phenyl, dimethylmethylene, dimethylsilicon, methylene, dihydrosilicon, or a bond.

In some embodiments, X₁ is N-phenyl and X₂ is O, C(O), B-phenyl, dimethylmethylene, dimethylsilicon, methylene, dihydrosilicon, or a bond.

In some embodiments, X₁ is C(O) and X₂ is N-phenyl, B-phenyl, O, S, or a bond.

In some embodiments, X₁ is O or S and X₂ is B-phenyl or C(O).

In some embodiments, the present invention may be represented as consisting of fragments α and β, connected by a silicon atom:

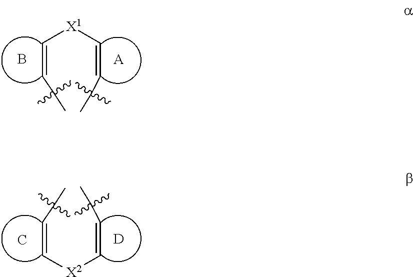

In some embodiments, fragments α and β are not identical.

In some embodiments, the molecule consisting of fragments α and β can be represented by the following structural formula:

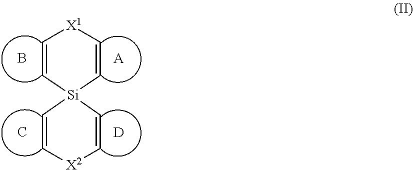

In the structure of formula (II):
X¹ and X² are independently selected from O, S, C(O), CR^{a}R^{b}, SiR^{a}R^{b}, NR^{c}, BR^{c}, or a bond. Each of A, B, C, and D is, independently, an optionally substituted six-membered aromatic or heteroaromatic ring, wherein at least one of rings A, B, C, and D contains at least one Nitrogen atom.
Each instance of R^{a}, R^{b}, and R^{c} is independently selected from H, a C₁-C₆ alkyl, a C₃-C₁₈ cycloalkyl, a C₆-C₁₈ aryl, a 5-20 atom heteroaryl, halo, or —CN.

In some embodiments at least one of X¹ and X² is not CH2.

In some embodiments, the molecule of formula (II) is not group symmetric. In some embodiments, the molecule of formula (II) is group symmetric.

In some embodiments, ring A, B, C, or D is substituted with one or more substituents selected from a C₁-C₆ alkyl, a C₃-C₁₈ cycloalkyl, a C₆-C₁₈ aryl, a 5-20 atom heteroaryl, halo, or —CN.

In some embodiments, at least one of rings A, B, C, and D contains at least one N. In some embodiments, at least two of rings A, B, C, and D contain at least one N. In some embodiments, at least three of rings A, B, C, and D contain at least one N. In some embodiments, each of rings A, B, C, and D contains at least one N.

In some embodiments, at least one of rings A, B, C, and D is a six-membered ring. In some embodiments, at least two of rings A, B, C, and D are six-membered rings. In some embodiments, at least three of rings A, B, C, and D are six-membered rings. In some embodiments, each of rings A, B, C, and D is a six-membered ring.

In some embodiments, each of rings A, B, C, and D contains 0, 1, or 2 Nitrogens. In some embodiments, each of rings A, B, C, and D contains 0 or 1 Nitrogen.

In some embodiments, the structure of formula (I) can be represented by the following structural formula:

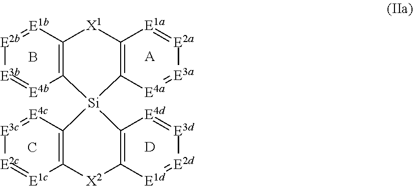

In the structure of formula (Ha):
Each atom E^{1a}, E^{2a}, E^{3a}, E^{4a}, E^{1b}, E^{2b}, E^{3b}, E^{4b}, E^{1c}, E^{2c}, E^{3c}, E^{4c}, E^{1d}, E^{2d}, E^{3d}, and E^{4d} is independently selected from CR^{d} or N.
Each instance of R^{d} is independently selected from H, a C₁-C₆ alkyl, a C₃-C₁₈ cycloalkyl, a C₆-C₁₈ aryl, a 5-20 atom heteroaryl, halo, or —CN.

In some embodiments, X¹ is SiR^{a}R^{b}, BR^{c}, O, or S. In some embodiments, X¹ is SiR^{a}R^{b} or BR^{c}.

In some embodiments, each instance of R^{a}, R^{b}, and R^{c} is independently selected from H, phenyl, and C₁₋₃ alkyl.

In some embodiments, each instance of R^{d} is H or C₁-C₃ alkyl.

In some embodiments, X₁ is B-phenyl and X₂ is O, S, N-phenyl, dimethylmethylene, dimethylsilicon, methylene, dihydrosilicon, or a bond.

In some embodiments, X₁ is N-phenyl and X₂ is O, C(O), B-phenyl, dimethylmethylene, dimethylsilicon, methylene, dihydrosilicon, or a bond.

In some embodiments, X₁ is C(O) and X₂ is N-phenyl, B-phenyl, O, S, or a bond.

In some embodiments, X₁ is O or S and X₂ is B-phenyl or C(O).

In some embodiments, the present invention is one of the compounds shown in Table 1.

In some embodiments, the present invention is an organic light-emitting device comprising a first electrode, a second electrode, and an organic layer between the first electrode and the second electrode. The organic layer comprises at least one light-emitting molecule selected from structural formulas (IA), (IB), (IC), or from the structural formulas in Table 1.

### Glossary

The term “alkyl,” as used herein, refers to a saturated aliphatic branched or straight-chain monovalent hydrocarbon radical having the specified total number of carbon atoms. Thus, “C₁-C₆ alkyl” means a radical having from 1-6 carbon atoms, inclusive of any substituents, in a linear or branched arrangement. Examples of “C₁-C₆ alkyl” include n-propyl, i-propyl, n-butyl, i-butyl, sec-butyl, t-butyl, n-pentyl, n-hexyl, 2-methylbutyl, 2-methylpentyl, 2-ethylbutyl, 3-methylpentyl, and 4-methylpentyl. An alkyl can be optionally substituted with halogen, —OH, C₁-C₆ alkyl, C₂-C₆ alkenyl, C₂-C₆ alkynyl, C₁-C₆ alkoxy, —NO₂, —CN, and —N(R′)(R²) wherein R¹ and R² are each independently selected from —H and C₁-C₃ alkyl.

The term “alkenyl,” as used herein, refers to a straight-chain or branched alkyl group having one or more carbon-carbon double bonds and having the specified total number of carbon atoms. Thus, “C₂-C₆ alkenyl” means a radical having 2-6 carbon atoms, inclusive of any substituents, in a linear or branched arrangement having one or more double bonds. Examples of “C₂-C₆ alkenyl” include ethenyl, propenyl, butenyl, pentenyl, hexenyl, butadienyl, pentadienyl, and hexadienyl. An alkenyl can be optionally substituted with the substituents listed above with respect to alkyl.

The term “alkynyl,” as used herein, refers to a straight-chain or branched alkyl group having one or more carbon-carbon triple bonds. Thus, “C₂-C₆ alkynyl” means a radical having 2-6 carbon atoms, inclusive of any substituents, in a linear or branched arrangement having one or more triple bonds. Examples of C₂-C₆ “alkynyl” include ethynyl, propynyl, butynyl, pentynyl, and hexynyl. An alkynyl can be optionally substituted with the substituents listed above with respect to alkyl.

The term “cycloalkyl,” as used herein, refers to a saturated monocyclic or fused polycyclic ring system containing from 3-12 carbon ring atoms. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2]bicyclooctane, decahydronaphthalene and adamantane. A cycloalkyl can be optionally substituted with the substituents listed above with respect to alkyl.

The term “amino,” as used herein, means an “—NH₂,” an “NHR^{p},” or an “NR^{p}R^{Q},” group, wherein R^{p} and R^{q}, each independently, can be C₁-C₁₂ alkyl, C₂-C₁₂ alkenyl, C₁-C₁₂ alkynyl, C₂-C₁₂ alkoxy, cycloalkyl, C₆-C₁₈ aryl, or 5-20 atom heteroaryl. Aminos may be primary (NH₂), secondary (NHRₚ) or tertiary (NRₚR_{q}).

The term “alkylamino,” as used herein, refers to an “NHRₚ,” or an “NRₚR_{q}” group, wherein R^{p} and R^{q} can be alkyl, alkenyl, alkynyl, alkoxy, or cycloalkyl. The term “dialkylamino,” as used herein, refers to an “NRₚR_{q}” group, wherein Rₚ and R_{q} can be alkyl, alkenyl, alkynyl, alkoxy, or cycloalkyl.

The term “alkoxy”, as used herein, refers to an “alkyl-O—” group, wherein alkyl is defined above. Examples of alkoxy group include methoxy or ethoxy groups. The “alkyl” portion of alkoxy can be optionally substituted as described above with respect to alkyl.

The term “aryl,” as used herein, refers to an aromatic monocyclic or polycyclic ring system consisting of carbon atoms. Thus, “C₆-C₁₈ aryl” is a monocylic or polycyclic ring system containing from 6 to 18 carbon atoms. Examples of aryl groups include phenyl, indenyl, naphthyl, azulenyl, heptalenyl, biphenyl, indacenyl, acenaphthylenyl, fluorenyl, phenalenyl, phenanthrenyl, anthracenyl, cyclopentacyclooctenyl or benzocyclooctenyl. An aryl can be optionally substituted with halogen, —OH, C₁-C₆ alkyl, C₂-C₆ alkenyl, C₂-C₆ alkynyl, C₁-C₆haloalkyl, C₁-C₆ alkoxy, C₆-C₁₈ aryl, C₆-C₁₈ haloaryl, (5-20 atom) heteroaryl, —C(O)C₁-C₃ haloalkyl, —C(O)—(C₆-C₁₈ aryl), —S(O)₂—, —NO₂, —CN, and oxo. In an example embodiment, if an aryl is substituted with C₆-C₁₈ aryl, C₆-C₁₈ haloaryl, or (5-20 atom) heteroaryl, those substituents are not themselves substituted with C₆-C₁₇ aryl, C₆-C₁₈ haloaryl, or (5-20 atom) heteroaryl.

The terms “halogen,” or “halo,” as used herein, refer to fluorine, chlorine, bromine, or iodine.

The term “heteroaryl,” as used herein, refers a monocyclic or fused polycyclic aromatic ring containing one or more heteroatoms, such as oxygen, nitrogen, or sulfur. For example, a heteroaryl can be a “5-20 atom heteroaryl,” which means a 5 to 20 membered monocyclic or fused polycyclic aromatic ring containing at least one heteroatom. Examples of heteroaryl groups include pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, purinyl, oxadiazolyl, thiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl. A heteroaryl can be optionally substituted with the same substituents listed above with respect to aryl.

In other embodiments, a “5-20 member heteroaryl” refers to a fused polycyclic ring system wherein aromatic rings are fused to a heterocycle. Examples of these heteroaryls include:

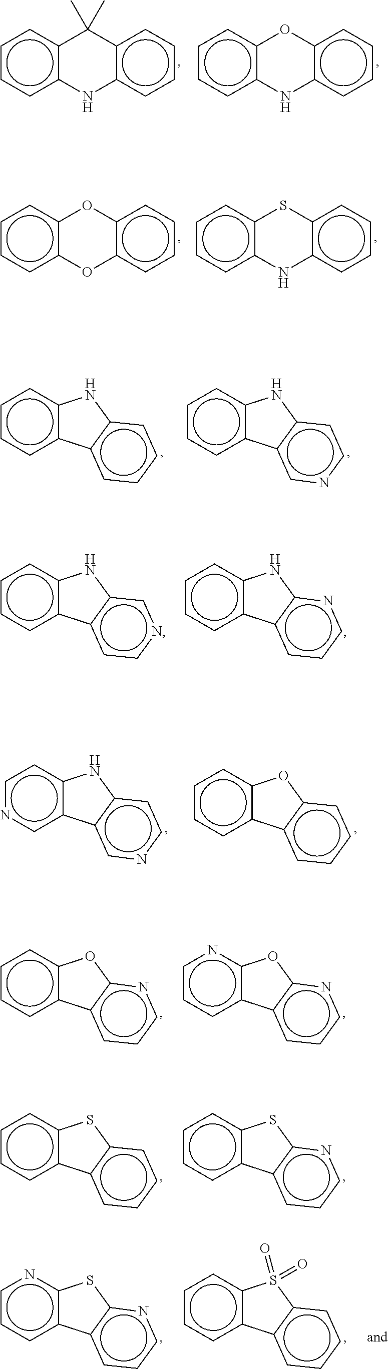

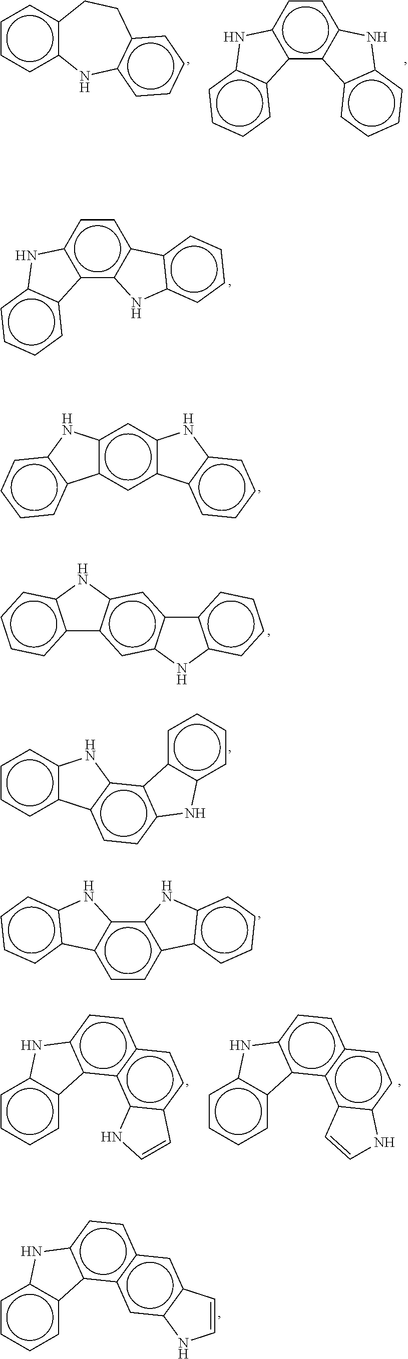

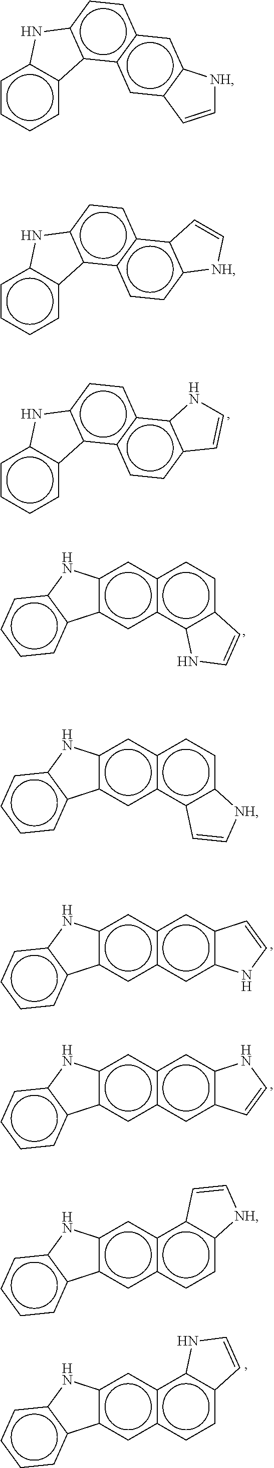

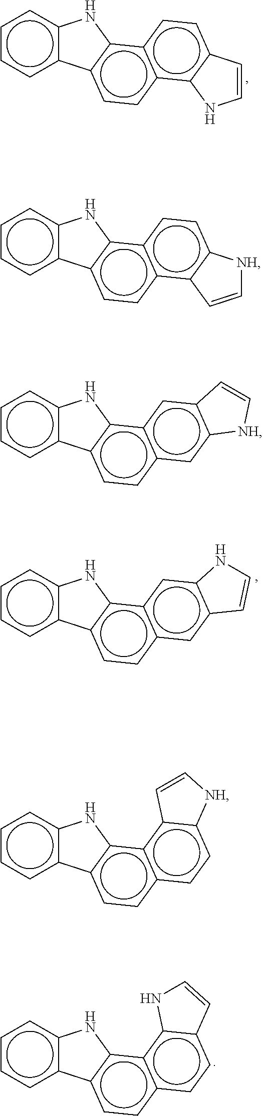

The term “haloalkyl,” as used herein, includes an alkyl substituted with one or more of F, Cl, Br, or I, wherein alkyl is defined above. The “alkyl” portion of haloalkyl can be optionally substituted as described above with respect to alkyl.

The term “haloaryl,” as used herein, includes an aryl substituted with one or more of F, Cl, Br, or I, wherein aryl is defined above. The “aryl” portion of haloaryl can be optionally substituted as described above with respect to aryl.

The term “oxo,” as used herein, refers to ═O.

The term “nitro,” as used herein, refers to —NO₂.

The term “symmetrical molecule,” as used herein, refers to molecules that are group symmetric or synthetic symmetric. The term “group symmetric,” as used herein, refers to molecules that have symmetry according to the group theory of molecular symmetry. The term “synthetic symmetric,” as used herein, refers to molecules that are selected such that no regioselective synthetic strategy is required.

The term “donor,” as used herein, refers to a molecular fragment that can be used in organic light emitting diodes and is likely to donate electrons from its highest occupied molecular orbital to an acceptor upon excitation. In an example embodiment, donors have an ionization potential greater than or equal to −6.5 eV.

The term “acceptor.” as used herein, refers to a molecular fragment that can be used in organic light emitting diodes and is likely to accept electrons into its lowest unoccupied molecular orbital from a donor that has been subject to excitation. In an example embodiment, acceptors have an electron affinity less than or equal to −0.5 eV.

The term “bridge,” as used herein, refers to a n-conjugated molecular fragment that can be included in a molecule which is covalently linked between acceptor and donor moieties. The bridge can, for example, be further conjugated to the acceptor moiety, the donor moiety, or both. Without being bound to any particular theory, it is believed that the bridge moiety can sterically restrict the acceptor and donor moieties into a specific configuration, thereby preventing the overlap between the conjugated ia system of donor and acceptor moieties. Examples of suitable bridge moieties include phenyl, ethenyl, and ethynyl.

The terms “acceptor”, “donor” and “bridge” are applied to fragments of a single molecule based on their relative electronic properties. A molecular fragment can be a donor in one molecule, but an acceptor in another molecule.

The term “multivalent,” as used herein, refers to a molecular fragment that is connected to at least two other molecular fragments. For example, a bridge moiety is multivalent.

“ [symbol] ” as used herein, refers to a point of attachment between two atoms.

### Principles of OLED

OLEDs are typically composed of a layer of organic materials or compounds between two electrodes, an anode and a cathode. The organic molecules are electrically conductive as a result of delocalization of t electronics caused by conjugation over part or all of the molecule. When voltage is applied, electrons from the highest occupied molecular orbital (HOMO) present at the anode flow into the lowest unoccupied molecular orbital (LUMO) of the organic molecules present at the cathode. Removal of electrons from the HOMO is also referred to as inserting electron holes into the HOMO. Electrostatic forces bring the electrons and the holes towards each other until they recombine and form an exciton (which is the bound state of the electron and the hole). As the excited state decays and the energy levels of the electrons relax, radiation is emitted having a frequency in the visible spectrum. The frequency of this radiation depends on the band gap of the material, which is the difference in energy between the HOMO and the LUMO.

As electrons and holes are fermions with half integer spin, an exciton may either be in a singlet state or a triplet state depending on how the spins of the electron and hole have been combined. Statistically, three triplet excitons will be formed for each singlet exciton. Decay from triplet states is spin forbidden, which results in increases in the timescale of the transition and limits the internal efficiency of fluorescent devices. Phosphorescent organic light-emitting diodes make use of spin-orbit interactions to facilitate intersystem crossing between singlet and triplet states, thus obtaining emission from both singlet and triplet states and improving the internal efficiency.

The prototypical phosphorescent material is iridium tris(2-phenylpyridine) (Ir(ppy)₃) in which the excited state is a charge transfer from the Ir atom to the organic ligand. Such approaches have reduced the triplet lifetime to about 1 μs, several orders of magnitude slower than the radiative lifetimes of fully-allowed transitions such as fluorescence. Ir-based phosphors have proven to be acceptable for many display applications, but losses due to large triplet densities still prevent the application of OLEDs to solid-state lighting at higher brightness.

Further, recent research suggests that traditional Iridium based OLEDs may have reached a physical performance limit. The brightness of an OLED will decrease as the time of decay increases. Since the highest energy triplet state is the origin of the luminescent transition in Ir-based materials, increasing the zero-field splitting through additional spin-orbit coupling will eventually lengthen the effective lifetime of the other two triplets. It is believed that this effect is responsible for the asymptote empirically observed at about 1 μs.

The recently developed thermally activated delayed fluorescence (TADF) seeks to minimize energetic splitting between singlet and triplet states (Δ). The reduction in exchange splitting from typical values of 0.4-0.7 eV to a gap of the order of the thermal energy (proportional to k_{B}T, where k_{B} represents the Boltzmann constant, and T represents temperature) means that thermal agitation can transfer population between singlet levels and triplet sublevels in a relevant timescale even if the coupling between states is small.

Example TADF molecules consist of donor and acceptor moieties connected directly by a covalent bond or via a conjugated linker (or “bridge”). A “donor” moiety is likely to transfer electrons from its HOMO upon excitation to the “acceptor” moiety. An “acceptor” moiety is likely to accept the electrons from the “donor” moiety into its LUMO. The donor-acceptor nature of TADF molecules results in low-lying excited states with charge-transfer character that exhibit very low A. Since thermal molecular motions can randomly vary the optical properties of donor-acceptor systems, a rigid three-dimensional arrangement of donor and acceptor moieties can be used to limit the non-radiative decay of the charge-transfer state by internal conversion during the lifetime of the excitation.

It is beneficial, therefore, to decrease energetic splitting between singlet and triplet states (Δ), and to create a system with increased reversed intersystem crossing (RISC) capable of exploiting triplet excitons. Such a system, it is believed, will result in decreased emission lifetimes. Systems with these features will be capable of emitting blue light without being subject to the rapid degradation prevalent in blue OLEDs known today.

### Compound of the Invention

The molecules of the present invention, when excited via thermal or electronic means, can produce light in the blue or green region of the visible spectrum. The molecules comprise molecular fragments including at least one donor moiety, at least one acceptor moiety, and optionally, a bridge moiety.

Electronic properties of the example molecules of the present invention can be computed using known ab initio quantum mechanical computations. By scanning a library of small chemical compounds for specific quantum properties, molecules can be constructed which exhibit the desired spin-orbit/thermally activated delayed fluorescence (SO/TADF) properties described above.

It could be beneficial, for example, to build molecules of the present invention using molecular fragments with a calculated triplet state above 2.75 eV. Therefore, using a time-dependent density functional theory using, as a basis set, the set of functions known as 6-31G* and a Becke, 3-parameter, Lee-Yang-Parr hybrid functional to solve Hartree-Fock equations (TD-DFT/B3LYP/6-31G*), molecular fragments (moieties) can be screened which have HOMOs above a specific threshold and LUMOs below a specific threshold, and wherein the calculated triplet state of the moieties is above 2.75 eV.

Therefore, for example, a donor moiety (“D”) can be selected because it has a HOMO energy (e.g., an ionization potential) of greater than or equal to −6.5 eV. An acceptor moiety (“A”) can be selected because it has, for example, a LUMO energy (e.g., an electron affinity) of less than or equal to −0.5 eV. The bridge moiety (“B”) can be a rigid conjugated system which can, for example, sterically restrict the acceptor and donor moieties into a specific configuration, thereby preventing the overlap between the conjugated a system of donor and acceptor moieties.

Accordingly, in a first aspect, the present invention is a molecule comprising at least one acceptor moiety A, at least one donor moiety D, and optionally, one or more bridge moieties B. The moiety D, for each occurrence independently, is a monocyclic or fused polycyclic aryl or heteroaryl having between 5 and 20 atoms, optionally substituted with one or more substituents. The moiety A, for each occurrence independently, is —CF₃, —CN, or a monocyclic or fused polycyclic aryl or heteroaryl having between 5 and 20 atoms, optionally substituted with one or more substituents. The moiety B, for each occurrence independently, is phenyl optionally substituted with one to four substituents. Each moiety A is covalently attached to either the moiety B or the moiety D, each moiety D is covalently attached to either the moiety B or the moiety A, and each moiety B is covalently attached to at least one moiety A and at least one moiety D. In an example embodiment of the first aspect, each moiety A is bonded either to moiety B or moiety D, each moiety B is bonded either to moiety A, moiety D. or a second moiety B, and each moiety D is bonded either to moiety A or moiety B. In another example embodiment of the first aspect, the moieties A are different than the moieties D.

The foregoing rules of connection mean that the moiety A cannot be connected to another moiety A, the moiety D cannot be connected to another moiety D. and that each moiety B is multivalent, and therefore must be connected to at least two other moieties, either a moiety A, a moiety D, or a second moiety B. It is understood that within a molecule no molecular fragment represented by A is the same as any molecular fragment represented by D.

In a second aspect, the present invention is a molecule comprising at least one acceptor moiety A, at least one donor moiety D, and optionally, one or more bridge moieties B, wherein A, D, and B are defined above with respect to the first aspect ofthe present invention. In addition to the moieties recited above in the first aspect, the moiety D can be —N(C₆-C₁₈aryl)₂. In addition to the moieties recited above with respect to the first aspect, the moiety A, can be —S(O)₂—. In addition to the moieties recited above with respect to the first aspect, the moiety B can be C₂-C₆ alkenyl, C₂-C₆ alkynyl, or C₅-C₁₂ cycloalkyl optionally substituted with one to four substituents.

In a third aspect, the present invention is a molecule defined by the structural formula (V)

(A)ₘ-(B)ₗ-(D)ₚ (V)

wherein A, B, and D are defined above with respect to the first and second aspects, and

the moiety D, for each occurrence independently, is optionally substituted with one or more substituents each independently selected from C₁-C₆ alkyl, C₂-C₆ alkenyl, C₂-C₆ alkynyl, C₆-C₁₈ aryl, (5-20 atom) heteroaryl, C₁-C₆ alkoxy, amino, C₁-C₃ alkylamino, C₁-C₃ dialkylamino, or oxo;

the moiety A, for each occurrence independently, is optionally substituted with one or more substituents independently selected from C₁-C₆ alkyl, C₂-C₆ alkenyl, C₂-C₆ alkynyl, C₆-C₁₈ aryl, (5-20 atom) heteroaryl, C₁-C₆ alkoxy, —C(O)C₁-C₃ haloalkyl, —S(O₂)H, —NO₂, —CN, oxo, halogen, or C₆-C₁₈ haloaryl:

the moiety B, for each occurrence independently, is optionally substituted with one to four substituents, each independently selected from C₁-C₆ alkyl, C₂-C₆ alkenyl, C₂-C₆ alkynyl, C₆-C₁₈ aryl, or (5-20 atom) heteroaryl:

m is an integer greater than 1;

p is an integer greater than 1; and

l is either 0 or an integer greater than one. In an example embodiment, l is greater than 1. In another example embodiment, l is 0, 1, or 2.

In a fourth aspect, the present invention is a molecule defined by the structural formula (V)

(A)ₘ-(B)ₗ-(D)ₚ (V)

wherein A, B, and D are defined above with respect to the first or second aspects of the present invention, and

the moiety D, for each occurrence independently, is optionally substituted, in addition to the substituents described above with respect to the third aspect of the present invention, with —N(C₆-C₁₈ aryl)₂;

the moiety A, for each occurrence independently, is optionally substituted as described above with respect to the third aspect of the present invention;

the moiety B, for each occurrence independently, is optionally substituted as described above with respect to the third aspect of the present invention;

m is an integer greater than 1;

p is an integer greater than 1; and

l is either 0 or an integer greater than one. In an example embodiment, l is greater than 1. In another example embodiment, l is 0, 1, or 2.

In a fifth aspect, the present invention is molecule defined by the structural formula (V)

(A)ₘ-(B)ₗ-(D)ₚ (V)

wherein A, B, and D are defined above with respect to the first and second aspects of the present invention, and

the moiety D, for each occurrence independently, is optionally substituted as described above with respect to the third and fourth aspects, and further wherein, each alkyl, alkenyl, alkynyl, aryl, and heteroaryl optionally further substituted with one or more substituents selected from C₁-C₆ alkyl, 5-20 atom heteroaryl, or —N(C₆-C₁₈aryl)₂;

the moiety A, for each occurrence independently, is optionally substituted as described above with respect to the third aspect of the present invention;

the moiety B, for each occurrence independently, is optionally substituted as described above with respect to the third aspect of the present invention;

m is an integer greater than 1;

p is an integer greater than 1; and

l is either 0 or an integer greater than one. In an example embodiment, l is greater than 1. In another example embodiment, l is 0, 1, or 2.

Structural formula (V) above can be linear or it can be branched.

### Combinatorial Assembly and Screening

Example molecules of the present invention having desirable properties, such as color of visible emission, can be constructed from the acceptor, donor, and bridge moieties described above using a combinatorial process described below. While only a few example compounds are illustrated below, it is understood that different combinations of different moieties can be used to create a combinatorial library of compounds. The example moieties below are intended only to illustrate the concepts herein, and are not intended to be limiting.

In the first step, a library of chemical moieties are screened for their abilities to function as acceptor or donor moieties. Example properties examined include desirable quantum mechanical computations such as the ionization potential of the highest occupied molecular orbital (i.e., a “donor” moiety) and the electron affinity of the lowest unoccupied molecular orbital (i.e., an “acceptor” moiety). In an example embodiment, a donor moiety can be selected if it is calculated that it has an ionization potential of greater than or equal to −6.5 eV. In another example embodiment, an acceptor moiety can be selected if it is calculated that it has an electron affinity of less than or equal to −0.5 eV. An example donor moiety selected after screening could be:

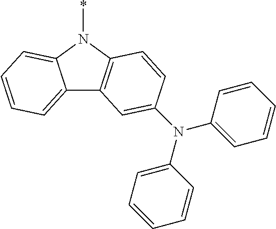 [SMILES: *n1c2ccccc2c2cc(N(c3ccccc3)c3ccccc3)ccc21]

and an example acceptor moiety selected after screening could be:

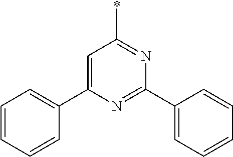 [SMILES: *c1cc(-c2ccccc2)nc(-c2ccccc2)n1]

wherein (*) represents a point of attachment for the donor and acceptor moieties either to each other or to a bridge moiety.

In a second, optional, step, if the selected donor and/or acceptor is “multi-site,” the multi-site donor moiety is combined with a single-site bridge moiety, and/or the multi-site acceptor moiety is combined with a single-site bridge moiety. If the donor and/or acceptor moieties are “single-site” moieties, then multi-site bridge moieties can be combined with the selected moieties. For the purposes of the combinatorial assembly, the number of “sites” refers to how many potentially different moieties can be attached. For example, the moiety below has one “site”:

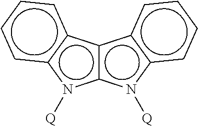

because all moieties attached at the position labeled Q must be the same. Similarly, the moiety below has two “sites” because Q and M can be the same or different:

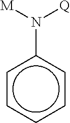

Thus, the nitrogen atom in the molecule is “multi-site.”

In the example moieties from the first step, both moieties are single-site. An example “multi-site” bridge could be:

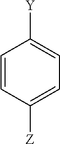

wherein the moieties attached at Y and Z are different. If the donor moiety combines with a bridge, and the acceptor combines with a bridge, the following moieties are created:

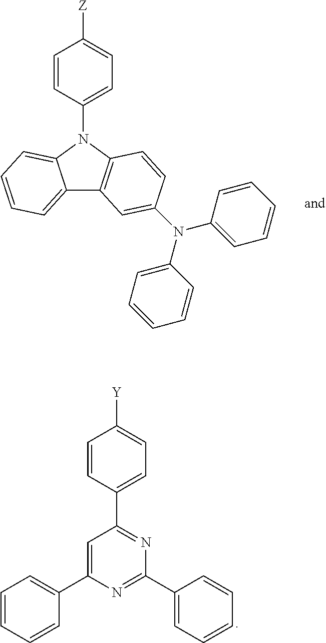

In a third step, the second step can be repeated to continuously add bridge moieties to the molecule. The only limitation is the size of final molecules that are going to be generated. The bridge molecules can be added at position Y or Z, indicated above, and can be the same bridge moiety, or a different bridge moiety. In one example embodiment, the number of bridge moieties can be limited to a number between 0 and 3. In another example, the number of donor moieties and acceptor moieties, or the total molecular weight of the molecule can be limited. In an example embodiment, the molecules are symmetrical. The symmetry can be used to limit the molecules in the combinatorial process to those that are stable. Therefore, for example, an additional bridge moiety added to the moieties from step two could be:

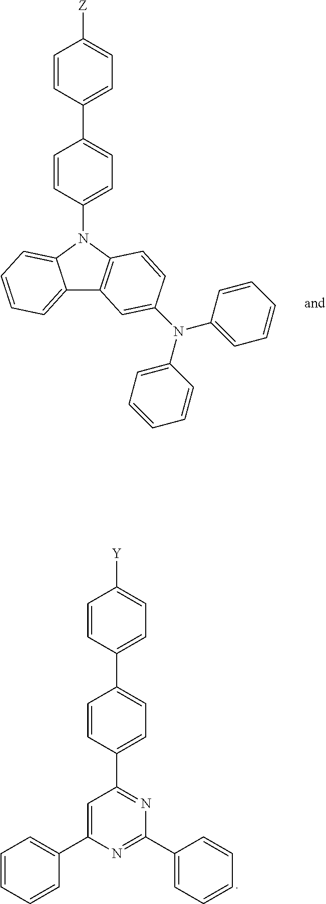

In a fourth step, the unattached point on the bridge moieties only combine with either (1) a donor moiety or an acceptor moiety that does not have a bridge moiety attached; or (2) other bridge moieties that is attached to either an acceptor moiety or a donor moiety such that the size limitation in step three is not violated, and that each molecule comprises at least one donor moiety and one acceptor moiety.

Using the example moieties and the rules described above, the following example molecules can be created:

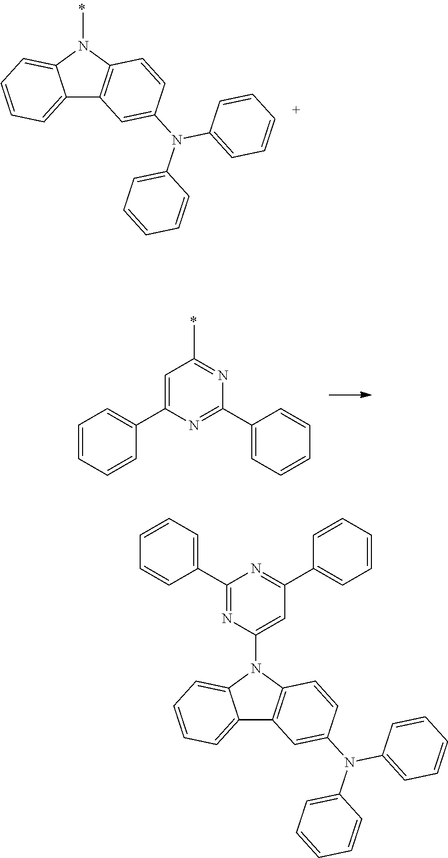 [SMILES: Cn1c2ccccc2c2cc(N(c3ccccc3)c3ccccc3)ccc21 ; Cc1cc(-c2ccccc2)nc(-c2ccccc2)n1 ; c1ccc(-c2cc(-n3c4ccccc4c4cc(N(c5ccccc5)c5ccccc5)ccc43)nc(-c3ccccc3)n2)cc1]

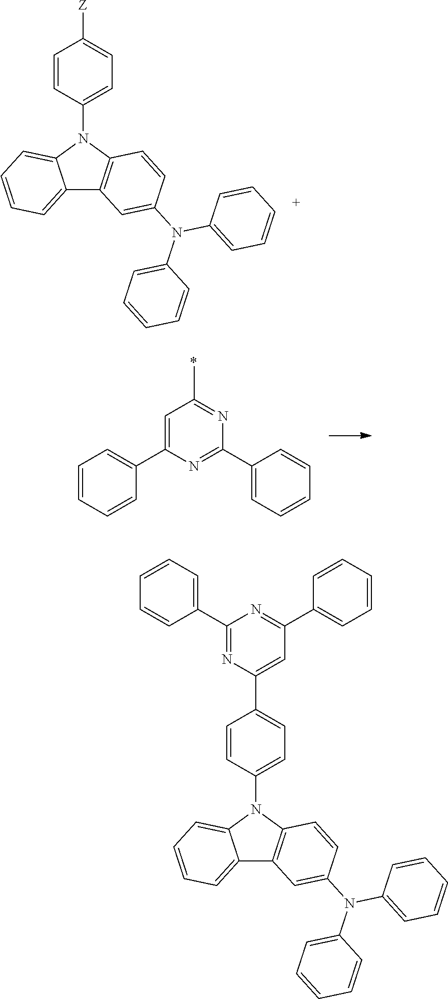

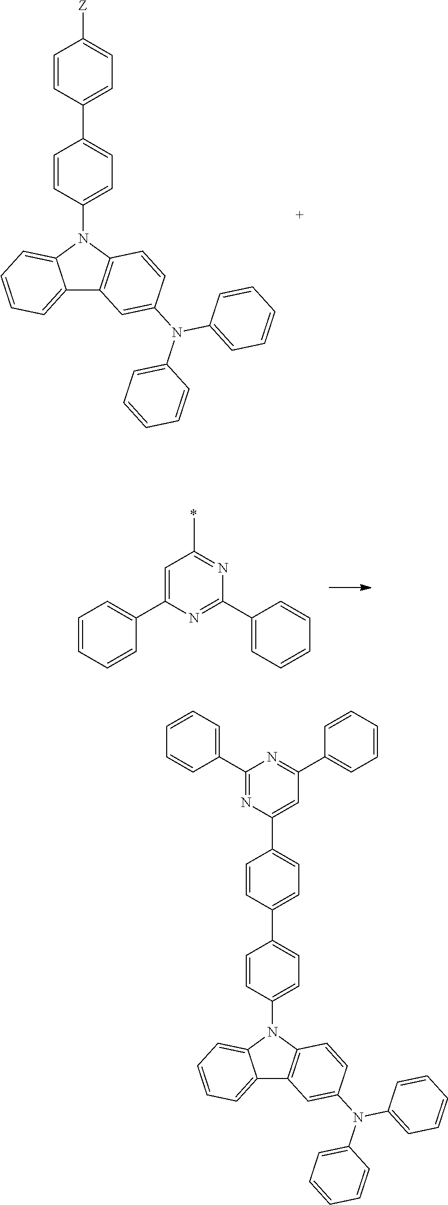

In the fifth step, the combined potential donors, acceptors, and bridges can be screened based on quantum mechanical computations such as desired HOMO and LUMO values, as well as vertical absorption (the energy required to excite the molecule from the ground state to the excited state), rate of decay (S1 to S0 oscillator strength, e.g., how fast and/or how bright the molecule's emission after excitation), estimated color of visible light emission in nanometers, and the singlet-triplet gap (the energy difference between the lowest singlet excited state, S1, the lowest triplet excited state, TI). Examples of the results of such calculations obtained for the molecules exemplified in the present application are provided in Table 1.

### Exemplification

It is understood that substituents and substitution patterns on the compounds of the invention can be selected by one of ordinary skill in the art to provide compounds that are chemically stable and that can be readily synthesized by techniques known in the art, as well as those methods set forth in Theophil Eicher, et al., The Chemistry of Heterocycles: Structures, Reactions, Synthesis, and Applications, which is incorporated herein by reference in its entirety.

An exemplary synthesis is represented by the following reaction scheme:

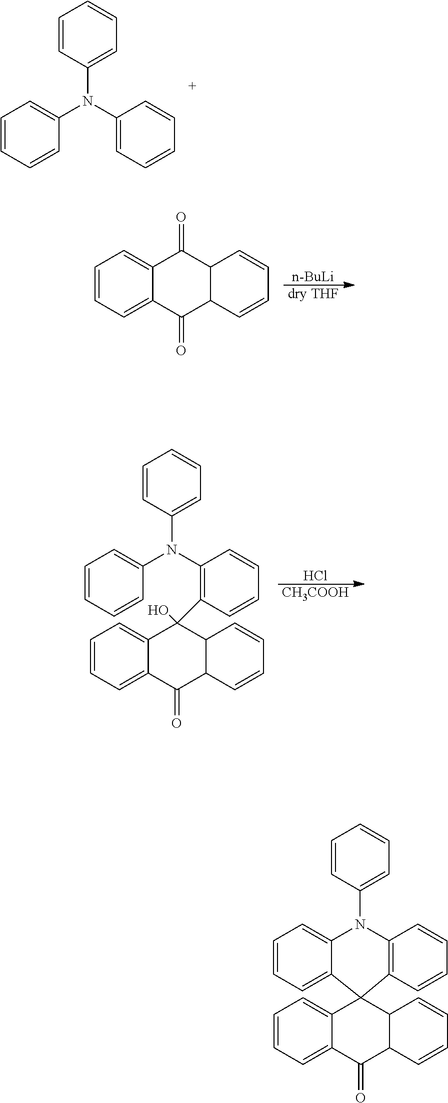

In this exemplary synthesis, n-BuLi (1.6 M in hexane, 14.6 mL, 23.3 mmol) is added to a solution of of 2-bromotriphenylamine (7.54 g, 23.3 mmol) in dry THF (180 mL) at −78° C. That mixture is stirred for 1.5 hours at −78° C. Anthraquinone (4.3 g, 21.2 mmol) is added to the reaction solution, which is then stirred for 1 day at 0° C. The reaction mixture is extracted into chloroform. The organic layer is dried over MgSO₄, filtered, and concentrated in vacuo, then purified by column chromatography. The reaction product (3.21 g, 7.09 mmol), acetic acid (55 mmol), and HCl (5.5 mL) are stirred for 4 hours under reflux. The reaction mixture is filtered, and the product is extracted into chloroform. The organic layer is dried over MgSO₄, filtered, and concentrated in vacuo, then purified by column chromatography.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A molecule represented by the following structural formula: 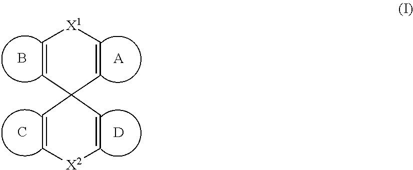
wherein:
X¹ and X² are independently selected from O, S, C(O), CR^{a}R^{b}, SiR^{a}R^{b}, NR^{c}, BR, or a bond, wherein at least one of X¹ and X² is not CH₂;
each of rings A, B, C, and D is, independently, an optionally substituted six-membered aromatic or heteroaromatic ring, wherein at least one of rings A, B, C, and D contains at least one nitrogen atom; and
each instance of R^{a}, R^{b}, and R^{c} is independently selected from H, a C₁-C₆ alkyl, a C₃-C₁₈ cycloalkyl, a C₆-C₈ aryl, a 5-20 atom heteroaryl, halo, or —CN.

2. The molecule of claim 1, represented by the following structural formula: 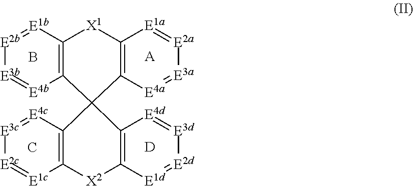
wherein:
each atom E^{1a}, E^{2a}, E^{3a}, E^{4a}, E^{1b}, E^{2b}, E^{3b}, E^{4b}, E^{1c}, E^{2c}, E^{3c}, E^{4c}, E^{1d}, E^{2d}, E^{3d}, and E^{4d} is independently selected from CR^{d} or N, provided that each ring A, B, C, and D contains 0, 1, or 2 nitrogen atoms; and
each instance of R^{d} is independently selected from H, a C₁-C₆ alkyl, a C₃-C₁₈ cycloalkyl, a C₆-C₁₈ aryl, a 5-20 atom heteroaryl, halo, or —CN.

3. The molecule of claim 1, wherein X¹ is SiR^{a}R^{b}, BR^{c}, O, or S.

4. The molecule of claim 3, wherein X¹ is SiR^{a}R^{b} or BR^{c}.

5. The molecule of claim 1, wherein each instance of R^{a}, R^{b}, and R^{c} is independently selected from H, phenyl, and C₁₋₃ alkyl.

6. The molecule of claim 1, wherein each instance of R^{d} is H or C₁-C₃ alkyl.

7. The molecule of claim 1, wherein each of rings A, B, C, and D contains no more than one nitrogen atom.

8. The molecule of claim 1, wherein at least two of rings A, B, C, and D contain at least one nitrogen atom.

9. The molecule of claim 8, wherein at least three of rings A, B, C, and D contain at least one nitrogen atom.

10. The molecule of claim 9, wherein each of rings A, B, C, and D contains at least one nitrogen atom.

11. The molecule of claim 1, wherein X₁ is B-phenyl and X₂ is O, S, N-phenyl, —C(CH₃)₂—, —Si(CH₃)₂—, —CH₂—, —SiH₂—, or a bond.

12. The molecule of claim 1, wherein X₁ is N-phenyl and X₂ is O, C(O), B-phenyl, —C(CH₃)₂—, —Si(CH₃)₂—, —CH₂—, —SiH₂—, or a bond.

13. The molecule of claim 1, wherein X₁ is C(O) and X₂ is N-phenyl, B-phenyl, O, S, or a bond.

14. The molecule of claim 1, wherein X₁ is O or S and X₂ is B-phenyl or C(O).

15. A molecule represented by the following structural formula: 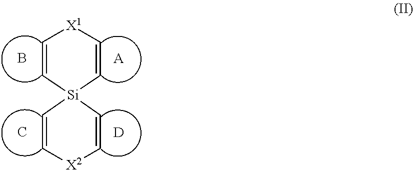
wherein:
X¹ and X² are independently selected from O, S, C(O), CR^{a}R^{b}, SiR^{a}R^{b}, NR^{c}, BR^{c}, or a bond, wherein at least one of X¹ and X² is not CH₂;
each of A, B, C, and D is, independently, an optionally substituted six-membered aromatic or heteroaromatic ring, wherein at least one of rings A, B, C, and D contains at least one Nitrogen atom;
each instance of R^{a}, R^{b}, and R^{c} is independently selected from H, a C₁-C₆ alkyl, a C₃-C₁₈ cycloalkyl, a C₆-C₁₈ aryl, a 5-20 atom heteroaryl, halo, or —CN.

16. A molecule represented by any one structural formula as shown in Table I.

17. An organic light-emitting device containing:
a first electrode;
a second electrode; and
an organic layer disposed between the first electrode and the second electrode, wherein the organic layer comprises at least one molecule as defined by claim 1.
